SEQUENCE LISTING (28 sequences)

SEQ ID NO: 1 - protein, 329 aa, Artificial Sequence
  Synthetic polymer.
MRGSHHHHHHGMASMTGGQQMGRDLYDDDDKDPMAQVINTNSLSLLTQNNLNKSQSSLSSAIERLSSGLRINSAKDDAAGQAIANRFTSNIKGLTQASRNANDGISIAQTTEGALNEINNNLQRVRELSVQATNGTNSDSDLKSIQDEIQQRLEEIDRVSNQTQFNGVKVLSQDNQMKIQVGANDGETITIDLQKIDVKSLGLDGFNVNSPGISGGGGGILDSMGTLINEDAAAAKKSTANPLASIDSALSKVDAVRSSLGAIQNRFDSAITNLGNTVTNLNSARSRIEDADYATEVSNMSKAQILQQAGTSVLAQANQVPQNVLSLLR

SEQ ID NO: 2 - protein, 275 aa, Artificial Sequence
  Synthetic polymer.
MSGLRINSAKDDAAGQAIANRFTSNIKGLTQASRNANDGISIAQTTEGALNEINNNLQRVRELSVQATNGTNSDSDLKSIQDEIQQRLEEIDRVSNQTQFNGVKVLSQDNQMKIQVGANDGETITIDLQKIDVKSLGLDGFNVNSPGISGGGGGILDSMGTLINEDAAAAKKSTANPLASIDSALSKVDAVRSSLGAIQNRFDSAITNLGNTVTNLNSARSRIEDADYATEVSNMSKAQILQQAGTSVLAQANQVPQNVLSLLVPRGSHHHHHHG

SEQ ID NO: 3 - protein, 233 aa, Artificial Sequence
  Synthetic polymer.
MSGLRINSAKDDAAGQAIANRFTSNIKGLTQASRNANDGISIAQTTEGALNEINNNLQRVRELSVQATNGTNSDSDLKSIQDEIQQRLEEIDRVSNQTQFNGVKVLSQDNQMKIQVGANDGETITIDLQKIDVKSLGLDGFNVNSPGSTANPLASIDSALSKVDAVRSSLGAIQNRFDSAITNLGNTVTNLNSARSRIEDADYATEVSNMSKAQILQQAGLVPRGSHHHHHHG

SEQ ID NO: 4 - protein, 250 aa, Artificial Sequence
  Synthetic polymer.
MSGLRINSAKDDAAGQAIANRFTSNIKGLTQASRNAADGISIAQTTEGALNEINNNLQRVRELSVQATAGANADAALKAIQAEIQQRLEEIDRVSQQTQAAAVKVLSQDNAMAIQVGANDGAAITIDLQKIDVKSLGLDGFNVNSPGSTANPLASIDSALSKVDAVRSSLGAIQNRFDSAITNLGNTVTNLNSARSRIEDADYATEVSQMSKAQILQQAGTSVLAQANQVPQNVLSLLVPRGSHHHHHHG

SEQ ID NO: 5 - protein, 300 aa, Artificial Sequence
  Synthetic polymer.
MRGSHHHHHHGMASMTGGQQMGRDLYDLVPRGSAKDPSGLRINSAKDDAAGQAIANRFTSNIKGLTQASRNANDGISIAQTTEGALNEINNNLQRVRELSVQATNGTNSDSDLKSIQDEIQQRLEEIDRVSNQTQFNGVKVLSQDNQMKIQVGANDGETITIDLQKIDVKSLGLDGFNVNSPGISGGGGGILDSMGTLINEDAAAAKKSTANPLASIDSALSKVDAVRSSLGAIQNRFDSAITNLGNTVTNLNSARSRIEDADYATEVSNMSKAQILQQAGTSVLAQANQVPQNVLSLLR

SEQ ID NO: 6 - protein, 250 aa, Artificial Sequence
  Synthetic polymer.
MSGLRINSAKDDAAGQAIANRFTSNIKGLTQASRNANDGISIAQTTEGALNEINNNLQRVRELSVQATNGTNSDSDLKSIQDEIQQRLEEIDRVSNQTQFNGVKVLSQDNQMKIQVGANDGETITIDLQKIDVKSLGLDGFNVNSPGSTANPLASIDSALSKVDAVRSSLGAIQNRFDSAITNLGNTVTNLNSARSRIEDADYATEVSNMSKAQILQQAGTSVLAQANQVPQNVLSLLVPRGSHHHHHHG

SEQ ID NO: 7 - protein, 293 aa, Artificial Sequence
  Synthetic polymer.
MGHHHHHHSGMEEFNMRINTNVAAMNTYSRLTAANTAKSNSLAKLSSGLRINKAGDDAAGLAISEKMKSQIGGLTQAKRNAQDGISLVQTAEGALNETHSILERMRDLAVQGSNGTLTSSDRGSINKELKALHQELTRISNTTEFNTQKLFSQTKQKSVTFTFQIGANAGQTLSVAITAMSGEALLVSTDAKFSLNAAGTNAGAMIKSIDAAIAKVSDQRADLGAVQNRLEHTINNLTATNENLSDANSRIRDVDMAEEMMTFTKSNILSQAATSMLAQANAMPNSVLNLLQG

SEQ ID NO: 8 - protein, 280 aa, Artificial Sequence
  Synthetic polymer.
MGHHHHHHSGMRINHNISALNAWRNIDQTQYSMSKTLERLSSGLRINRAGDDAAGLAISEKMRGQIKGLNMAIKNAQDAISLIQTAEGALTEVHSILQRMRELAVQAASDTNTNVDREQIQKEIDQLREEIDRIARTTEFNTKKLLDGKLEGFRSQVDAKVVTGGNINVQLGTVSSKAVEGTYVIEVGAAERAIMVVDAAIHRVSTARAALGAIQNRLEHTISNLGVAAENLTAAESRIRDADMAKEMMEFTKQQILLQSSMAMLAQSNTLPQNVLQLMR

SEQ ID NO: 9 - protein, 168 aa, Artificial Sequence
  Synthetic polymer.
MGHHHHHHSGLNMAIKNAQDAISLIQTAEGALTEVHSILQRMRELAVQAASDTNTNVDREQIQKEIDQLREEIDRIARTTEFNTKKLLDGKLEGFRSQVDAKVVTGGNINVQLGTVSSKAVEGTYVIEVGAAERAIMVVDAAIHRVSTARAALGAIQNRLEHTISNLG

SEQ ID NO: 10 - protein, 285 aa, Artificial Sequence
  Synthetic polymer.
MGHHHHHHSGMSLRINNNIEALNAWRALNSTSNALQKSMEKLSSGLRINRAGDDAAGLAISEKLRAQIRGLNQAIRNAQDGISLIQTAEGGLSEIQNILQRMRELGVQAANGTLNNQDISAITTELNQLFNEIDRIAGATEFNTKNLLAVSTGLVVTLQVGANAGQVIAFTIDNAGTASLGLSSADLAINDNASASAFISKVDSALQKVSTYRANLGSIQNRLEHTIANLGIASENLSASESRIRDVDMAAEMMNFTKNQILQQAGVAILAQANQAPQAVLQLLR

SEQ ID NO: 11 - protein, 171 aa, Artificial Sequence
  Synthetic polymer.
MGHHHHHHSGLNQAIRNAQDGISLIQTAEGGLSEIQNILQRMRELGVQAANGTLNNQDISAITTELNQLFNEIDRIAGATEFNTKNLLAVSTGLVVTLQVGANAGQVIAFTIDNAGTASLGLSSADLAINDNASASAFISKVDSALQKVSTYRANLGSIQNRLEHTIANLG

SEQ ID NO: 12 - protein, 146 aa, Artificial Sequence
  Synthetic polymer.
MGHHHHHHSGLNQAIRNAQDGISLIQTAEGGLSEIQNILQRMRELGVQAANGTLNNQDISAITTELNQLFNEIDRIAGATEFNTKNLLAAGTASLGLSSADLAINDNASASAFISKVDSALQKVSTYRANLGSIQNRLEHTIANLG

SEQ ID NO: 13 - protein, 116 aa, Artificial Sequence
  Synthetic polymer.
MGHHHHHHSASAFISKVDSALQKVSTYRANLGSIQNRLEHTIANLGPDGLNQAIRNAQDGISLIQTAEGGLSEIQNILQRMRELGVQAANGTLNNQDISAITTELNQLFNEIDRIA

SEQ ID NO: 14 - protein, 103 aa, Artificial Sequence
  Synthetic polymer.
MGHHHHHHSNNQDISAITTELNQLFNEIDRIAGATGSGGLSEIQNILQRMRELGVQAANGTLNGGSASAFISKVDSALQKVSTYRANLGSIQNRLEHTIANLG

SEQ ID NO: 15 - protein, 170 aa, Artificial Sequence
  Synthetic polymer.
MGHHHHHHSGLAQASRNAQDAISIAQTAEGALDETQSILQRVRELGVQGANGTLTADDINALQAEVDQLIAEIDRIAGATEFNTQNLLDGSFTTKAFQVGANSGQNMTLTIGKMDTTTLGLSSADLAINDNAFANGAISTVDSALQKVSAERAKLGAIQNRLEHTIANLG

SEQ ID NO: 16 - protein, 170 aa, Artificial Sequence
  Synthetic polymer.
MGHHHHHHSGLAQASRQAQDAISIAQTAEGALDETQSILQRVRELGVQGADGTLTADDIDALQAEVDQLIAEIDRIAGATEFATQKLLDGSFTTKAFQVGAASGQDVTLTIGKVDTTTLGLSSADLAIDSAAFADGAISTVDSALQKVSAERAKLGAIQNRLEHTIAQLG

SEQ ID NO: 17 - protein, 274 aa, Artificial Sequence
  Synthetic polymer.
MRGSHHHHHHGMASMTGGQQMGRDLYDDDDKDPMAQVINTNSLSLLTQNNLNKSQSSLSSAIERLSSGLRINSAKDDAAGQAIANRFTSNIKGLTQASRNANDGISIAQTTEGALNEINNNLQRVRELSVQATNGTNSDSDLKSIQDEIQQRLEEIDRVSNQTQFNGVKVLSQDNQMKIQVGANDGETITIDLQKIDVKSLGLDGFNVNSPGISGGGGGILDSMGTLINEDAAAAKKSTANPLASIDSALSKVDAVRSSLGAIQNRFDSAITNL

SEQ ID NO: 18 - protein, 276 aa, Artificial Sequence
  Synthetic polymer.
MRGSHHHHHHGMASMTGGQQMGRDLYDDDDKDPFTSNIKGLTQASRNANDGISIAQTTEGALNEINNNLQRVRELSVQATNGTNSDSDLKSIQDEIQQRLEEIDRVSNQTQFNGVKVLSQDNQMKIQVGANDGETITIDLQKIDVKSLGLDGFNVNSPGISGGGGGILDSMGTLINEDAAAAKKSTANPLASIDSALSKVDAVRSSLGAIQNRFDSAITNLGNTVTNLNSARSRIEDADYATEVSNMSKAQILQQAGTSVLAQANQVPQNVLSLLR

SEQ ID NO: 19 - protein, 323 aa, Artificial Sequence
  Synthetic polymer.
MRGSHHHHHHGMASMTGGQQMGRDLYDDDDKDPMAQVINTNSLSLLTQNNLNKSQSSLSSAIERLSSGLRINSAKDDAAGQAIANRFTSNIKGLTQASRNANDGISIAQTTEGALNEINNNLQRVRELSVQATNGTNSDSDLKSIQDEIQQRLEEIDRVSNQTQFNGVKVLSQDNQMKIQVGANDGETITIDLQKIDVKSLGLIPGISGGGGGILDSMGTLINEDAAAAKKSTANPLASIDSALSKVDAVRSSLGAIQNRFDSAITNLGNTVTNLNSARSRIEDADYATEVSNMSKAQILQQAGTSVLAQANQVPQNVLSLLR

SEQ ID NO: 20 - protein, 270 aa, Artificial Sequence
  Synthetic polymer.
MRGSHHHHHHGMASMTGGQQMGRDLYDDDDKDPFTSNIKGLTQASRNANDGISIAQTTEGALNEINNNLQRVRELSVQATNGTNSDSDLKSIQDEIQQRLEEIDRVSNQTQFNGVKVLSQDNQMKIQVGANDGETITIDLQKIDVKSLGLIPGISGGGGGILDSMGTLINEDAAAAKKSTANPLASIDSALSKVDAVRSSLGAIQNRFDSAITNLGNTVTNLNSARSRIEDADYATEVSNMSKAQILQQAGTSVLAQANQVPQNVLSLLR

SEQ ID NO: 21 - protein, 316 aa, Artificial Sequence
  Synthetic polymer.
MRGSHHHHHHGMASMTGGQQMGRDLYDDDDKDPMAQVINTNSLSLLTQNNLNKSQSSLSSAIERLSSGLRINSAKDDAAGQAIANRFTSNIKGLTQASRNANDGISIAQTTEGALNEINNNLQRVRELSVQATNGTNSDSDLKSIQDEIQQRLEEIDRVSNQTQFNGVKVLSQDNQMKIQVGANDGETITIDLQKIIPGISGGGGGILDSMGTLINEDAAAAKKSTANPLASIDSALSKVDAVRSSLGAIQNRFDSAITNLGNTVTNLNSARSRIEDADYATEVSNMSKAQILQQAGTSVLAQANQVPQNVLSLLR

SEQ ID NO: 22 - protein, 263 aa, Artificial Sequence
  Synthetic polymer.
MRGSHHHHHHGMASMTGGQQMGRDLYDDDDKDPFTSNIKGLTQASRNANDGISIAQTTEGALNEINNNLQRVRELSVQATNGTNSDSDLKSIQDEIQQRLEEIDRVSNQTQFNGVKVLSQDNQMKIQVGANDGETITIDLQKIIPGISGGGGGILDSMGTLINEDAAAAKKSTANPLASIDSALSKVDAVRSSLGAIQNRFDSAITNLGNTVTNLNSARSRIEDADYATEVSNMSKAQILQQAGTSVLAQANQVPQNVLSLLR

SEQ ID NO: 23 - protein, 268 aa, Artificial Sequence
  Synthetic polymer.
MRGSHHHHHHGMASMTGGQQMGRDLYDDDDKDPMAQVINTNSLSLLTQNNLNKSQSSLSSAIERLSSGLRINSAKDDAAGQAIANRFTSNIKGLTQASRNANDGISIAQTTEGALNEINNNLQRVRELSVQATNGTNSDSDLKSIQDEIQQRLEEIDRVSNQTQFNGVKVLSQDNQMKIQVGANDGETITIDLQKIDVKSLGLIPGISGGGGGILDSMGTLINEDAAAAKKSTANPLASIDSALSKVDAVRSSLGAIQNRFDSAITNL

SEQ ID NO: 24 - protein, 261 aa, Artificial Sequence
  Synthetic polymer.
MRGSHHHHHHGMASMTGGQQMGRDLYDDDDKDPMAQVINTNSLSLLTQNNLNKSQSSLSSAIERLSSGLRINSAKDDAAGQAIANRFTSNIKGLTQASRNANDGISIAQTTEGALNEINNNLQRVRELSVQATNGTNSDSDLKSIQDEIQQRLEEIDRVSNQTQFNGVKVLSQDNQMKIQVGANDGETITIDLQKIIPGISGGGGGILDSMGTLINEDAAAAKKSTANPLASIDSALSKVDAVRSSLGAIQNRFDSAITNL

SEQ ID NO: 25 - protein, 282 aa, Artificial Sequence
  Synthetic polymer.
MRGSHHHHHHGMASMTGGQQMGRDLYDDDDKDPMAQVINTNSLSLLTQNNLNKSQSSLSSAIERLSSGLRINSAKDDAAGQAIANRFTSNIKGLTQASRNANDGISIAQTTEGALNEINNNLQRVRELSVQATNGTNSDSDLKSIQDEIQQRLEEIDRVSNQIPGISGGGGGILDSMGTLINEDAAAAKKSTANPLASIDSALSKVDAVRSSLGAIQNRFDSAITNLGNTVTNLNSARSRIEDADYATEVSNMSKAQILQQAGTSVLAQANQVPQNVLSLLR

SEQ ID NO: 26 - protein, 229 aa, Artificial Sequence
  Synthetic polymer.
MRGSHHHHHHGMASMTGGQQMGRDLYDDDDKDPFTSNIKGLTQASRNANDGISIAQTTEGALNEINNNLQRVRELSVQATNGTNSDSDLKSIQDEIQQRLEEIDRVSNQIPGISGGGGGILDSMGTLINEDAAAAKKSTANPLASIDSALSKVDAVRSSLGAIQNRFDSAITNLGNTVTNLNSARSRIEDADYATEVSNMSKAQILQQAGTSVLAQANQVPQNVLSLLR

SEQ ID NO: 27 - protein, 505 aa, Artificial Sequence
  Synthetic polymer.
MAQVINTNSLSLLTQNNLNKSQSSLSSAIERLSSGLRINSAKDDAAGQAIANRFTSNIKGLTQASRNANDGISIAQTTEGALNEINNNLQRVRELSVQATNGTNSDSDLKSIQDEIQQRLEEIDRVSNQTQFNGVKVLSQDNQMKIQVGANDGETITIDLQKIDVKSLGLDGFNVNGPKEATVGDLKSSFKNVTGYDTYAAGADKYRVDINSGAVVTDAAAPDKVYVNAANGQLTTDDAENNTAVDLFKTTKSTAGTAEAKAIAGAIKGGKEGDTFDYKGVTFTIDTKTGDDGNGKVSTTINGEKVTLTVADIATGAADVNAATLQSSKNVYTSVVNGQFTFDDKTKNESAKLSDLEANNAVKGESKITVNGAEYTANATGDKITLAGKTMFIDKTASGVSTLINEDAAAAKKSTANPLASIDSALSKVDAVRSSLGAIQNRFDSAITNLGNTVTNLNSARSRIEDADYATEVSNMSKAQILQQAGTSVLAQANQVPQNVLSLLR

SEQ ID NO: 28 - protein, 239 aa, Artificial Sequence
  Synthetic polymer.
MSGLRINSAKDDAAGQAAANRATSNIKGLTQASRNAADGISIAQTTEGALNEINNNLQRVRELSVQATAGANADAALKAIQAEIQQRLEEIDRVSQQTQAAAVKVLSQDNAMAIQVGANDGAAITIDLQKIDVKSLGLDGFNVNSPGSTANPLASIDSALSKVDAVRSSLGAIQNRFDSAITNLGNTVTNLNSARSRIEDADYATEVSQMSKAQILDQAGTSTLAQLVPRGSHHHHHHG

(19) United States
(12) Patent Application Publication
(10) Pub. No.: US 2022/0288198 A1
(43) Pub. Date: Sep. 15, 2022

(54) METHODS OF INCREASING VACCINE EFFICACY

(71) Applicant: Genome Protection, Inc., Buffalo, NY (US)

(72) Inventors: Andrei GUDKOV, Buffalo, NY (US); Ekaterina ANDRIANOVA, Buffalo, NY (US)

(21) Appl. No.: 17/638,321

(22) PCT Filed: Aug. 28, 2020
(86) PCT No.: PCT/US20/48425

§ 371 Date: Feb. 25, 2022

Related U.S. Application Data
(60) Provisional application No. 62/894,355, filed on Aug. 30, 2019.

(51) Int. Cl. A61K 39/39; C07K 14/255; A61P 37/04

(52) U.S. Cl. CPC A61K 39/39; C07K 14/255; A61P 37/04; A61K 2039/55516

(57) ABSTRACT

The present invention relates, in part, to compositions and methods for enhancement of an immune response and for increased vaccine efficacy by stimulation of the TLR5 receptor, for example, with a recombinant TLR5 agonist (e.g., a flagellin-based agent or variant thereof).

### CROSS-REFERENCE TO RELATED APPLICATIONS

This application claims the benefit of, and priority to, U.S. Provisional Application No. 62/894,355, filed Aug. 30, 2019, the contents of which are herein incorporated by reference in their entirety.

### FIELD OF THE INVENTION

The present disclosure relates to compositions and methods for improving and/or increasing vaccine efficacy.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: GPI-004PC ST25.txt; date recorded: Aug. 28, 2020; file size: 63,471 bytes).

### BACKGROUND

Due to immunosenescence related to aging, vaccination within the geriatric population has not been as efficacious as within the general population. Goodwin et al. Vaccine (2006) 24(8):1159-69 reported that the influenza vaccine had only a 17-53% clinical efficacy in the elderly while on the other hand displayed 70-90% clinical efficacy in young adults. This is a result of vaccinations' inability to produce an adequate immune response in older individuals due to age-related changes in both the nonspecific (innate) and specific (adaptive) immune response. Weinburger et al. Clin Infect Dis (2008) 46(7):1078-84 summarizes the changes in vaccine response with age as an increase in the threshold for induction of an antibody response to a vaccine. Older individuals often produce insufficient antibody titers required to booster critical vaccinations. This inadequate antibody response can be life threatening, particularly more so in the aging population with other comorbid diseases. Compromises to their ability to resist the influenza virus, the most common cause of pneumonia infections, has led to hospitalizations, lengthened hospital stays and an estimated 10,000 to 14,000 deaths in the United States during the 2015-2016 flu season in the over 65-year-old population (Rolfes et al. Influenza Other Respir Viruses. (2018) 12(1):132-137).

Limitations of current and developing vaccines can be overcome by increasing the initial or boosted antibody titers thereby increasing the antibody titers above the threshold required for efficacy. In addition, due to public scrutiny and confusion regarding a belief that aluminum in vaccines causes various disorders in recipients, a reduction in or elimination of the use of aluminum as an adjuvant in vaccines is preferable.

As such, therapies and/or preventative measures are needed in order to enhance the immune response to various stimuli in the geriatric population due to increased life expectancy and thus, risk of infection.

### SUMMARY OF THE INVENTION

Accordingly, the present invention provides, in certain aspects, methods of improving and/or increasing vaccine efficacy in a patient by administering to said patient a TLR5 agonist, such as entolimod (i.e., SEQ ID NO: 1) or variant thereof, in combination with an antigen that is a constituent of an infectious agent selected from a live and attenuated, killed, inactivated, and toxoid infectious agent. In some embodiments, the patient is a middle-aged patient or a geriatric patient. In further embodiments, the patient is immunosenescent. In embodiments, the patient's immune response is enhanced or promoted.

In some aspects, the present invention contemplates methods of promoting an immune response to an antigen in a patient in need thereof by administering to said patient a TLR5 agonist, such as entolimod or variant thereof. In embodiments, the patient's immune response is enhanced or promoted.

In some aspects, the present invention provides, in part, methods of preventing and/or reducing incidence of pneumonia infections (e.g., influenza) in a patient by administering a TLR5 agonist, such as entolimod or variant thereof, in combination with a vaccine to said patient, wherein, in some embodiments, the patient is a geriatric patient. In some embodiments, the vaccine is an influenza virus vaccine.

In various embodiments, the patient receives the TLR5 agonist and an immune response is enhanced or promoted before the patient receives the antigen or vaccine administration.

In various embodiments, the patient is middle-aged (e.g., between the ages of about 36 and 64 years old) or geriatric (equal to or greater than about 65 years old). In some embodiments, the patient is immunosenescent. In some embodiments, the patient has an impaired immune system. In further embodiments, the patient is immunocompromised.

In some embodiments, the present invention provides methods for improving and/or increasing vaccine efficacy in a patient, as measured by an increase in the patient's innate and/or adaptive immune responses. In some embodiments, methods and compositions of the present invention for for improving and/or increasing vaccine efficacy in a patient include maintaining and/or increasing the patient's T cell populations (e.g., CD4+ and/or CD8+ T cell populations). In further embodiments, methods of the present invention provide for mitigation of age-related immunosenescence as measured by an increase or restoration of a patient's antigen-specific antibody titers (e.g., IgG, IgM and IgA).

In some embodiments, the present invention provides methods for improving and/or increasing vaccine efficacy in a patient, as measured by higher titer levels of antigen-specific antibodies as compared to titer levels of antigen-specific antibodies in patients that were not administered the TLR5 agonist. In some embodiments, the present invention provides methods for improving and/or increasing vaccine efficacy in a patient, as measured by an increase in the patient's innate immune response, as compared to the innate immune response of a patient that was not administered the TLR5 agonist. In further embodiments, the present invention provides methods for improving and/or increasing vaccine efficacy in a patient, as measured by an increase in the patient's adaptive immune response, as compared to the adaptive immune response of a patient that was not administered the TLR5 agonist. In some embodiments, the patient's innate immune response and adaptive immune response are increased, as compared to the innate and adaptive immune responses of a patient that was not administered the TLR5 agonist.

In some embodiments, the present invention provides methods for improving and/or increasing vaccine efficacy in a patient, as measured by an increase and/or restoration of the patient's T cell population(s), as compared to the T cell populations of a patient that was not administered the TLR5 agonist. For example, in further embodiments, the patient's T cells, including T cells selected from one or more of CD4+ effector T cells, CD8+ effector T cells, CD4+ memory T cells, CD8+ memory T cells, CD4+ central memory T cells, CD8+ central memory T cells, natural killer T cells, CD4+ helper cells (including, without limitation Th1, Th2, and Th17), and CD8+ cytotoxic cells, are increased and/or restored, as compared to the T cell populations of a patient that was not administered the TLR5 agonist.

In some embodiments, the present invention provides methods for improving and/or increasing vaccine efficacy in a patient, as measured by a reduction of vaccine dosage, relative to the vaccine dosage of a patient that was not administered the recombinant or synthetic TLR5 agonist. In some embodiments, the present invention provides methods for improving and/or increasing vaccine efficacy in a patient, as measured by a reduction of the frequency of vaccine dosing, relative to the frequency of vaccine dosing of a patient that was not administered the recombinant or synthetic TLR5 agonist.

In some aspects, the present invention provides for methods of enhancing vaccine efficacy by increasing and/or improving the immune response to an antigen. For example, the antigen may be, without limitation, a whole cell, a virus, a protein, a protein subunit or fragment. In further embodiments, the antigen which stimulates an immune response against a disorder is a constituent of an infectious agent selected from a live and attenuated, killed, inactivated, and toxoid infectious agent. In further embodiments, the antigen is associated with and/or stimulates an immune response against a tumor cell, a cell with damaged DNA, or a senescent cell. In particular, a TLR5 agonist (e.g., a flagellin-based agent), such as entolimod, can be used in combination with a vaccine against a viral or pathogenic agent, such as an influenza vaccine, pneumococcal vaccine, or HIV vaccine. More specifically, a TLR5 agonist can be used as described herein to enhance the immune response to a vaccine for any influenza strain, such as H1N1, H2N3, and B influenza subtypes.

In embodiments, the present invention contemplates that a vaccine adjuvant, such as alum, is not administered. In various embodiments, the recombinant or synthetic TLR5 agonist of the present invention is administered to the patient without an aluminum gel or salt selected from aluminum hydroxide, aluminum phosphate, and potassium aluminum sulfate, AS04 (which is composed of aluminum salt and monophosphoryl lipid A (MPL)), AS03 (α-tocopherol, squalene, and polysorbate 80 in an oil-in-water emulsion) and ALHYDROGEL.

### BRIEF DESCRIPTION OF THE DRAWINGS

The foregoing features of embodiments will be more readily understood by reference to the following detailed description, taken with reference to the accompanying drawings, in which:

FIG. 1 depicts a schematic illustration of the timeline of the immunization study with or without co-administration of entolimod (SEQ ID NO: 1). Immunization, serum collection, and sacrifice/sample collection points of time are shown for groups of male NIH Swiss mice treated with Prevnar13 vaccine with or without entolimod.

FIG. 2 shows levels of pneumococcus/Prevnar13-specific IgG (left panels) and IgM (right panels) antibody titers as measured by ELISA in serum samples collected on Day 12 or Day 27 after immunization on Day 1 (with a booster immunization given on Day 15). Mean antibody titers are shown. * indicates statistically significant differences as determined by Student's t-test. All other comparison between matched PBS-treated and entolimod-treated groups were not statistically significant (P>0.3). For the IgM antibody titer panels, in each set of two histograms, the left bar represents pre-boost measurement and the right bar represents post-boost measurement.

FIG. 3 depicts the percentage of lymph cells in a single-cell suspension of total spleen cells from mice that were 113 weeks old at the time of co-administration of Prevnar13 and entolimod (at 0.1 μg/mouse and 1.0 μg/mouse) or PBS.

FIG. 4A and FIG. 4B show both the percentage (FIG. 4A) and total number (FIG. 4B) of lymph cells in a single-cell suspension of spleen cells from mice that were 73 weeks old at the time of co-administration of Prevnar13 and entolimod (at 0.1 μg/mouse and 1.0 μg/mouse) or PBS.

FIG. 5A, FIG. 5B, FIG. 5C, FIG. 5D, and FIG. 5E depict percentages and number of various T cell markers (e.g., CD8-, CD4+, and CD44+) in a single-cell suspension of spleen cells from mice that were 30 weeks old at the time of co-administration of Prevnar13 and entolimod (at 0.1 μg/mouse and 1.0 μg/mouse) or PBS.

FIG. 6 shows a schematic illustration of the immunization schedule of mice immunized with Tdap with and without entolimod. Mice were immunized on Day 1 of the experiment and received a booster immunization on Day 32. Serum samples were collected on Day 0 (baseline), Day 14, Day 28, and Day 46.

FIG. 7 depicts the results of the study co-administering entolimod with Tdap vaccine at different administration routes and formulations in order to determine whether vaccination efficacy was increased via measurement of tetanus-specific serum IgG levels. Mean antibody titers are shown.

FIG. 8 shows increased IgG titer measurements post-boost in the +3 days and +7 days vaccine administration groups, as compared to control groups.

### DETAILED DESCRIPTION OF THE INVENTION

The present invention relates, in part, to compositions and methods for enhancing an immune response and/or increasing vaccine efficacy in a patient by stimulation of the TLR5 receptor, for example, with a recombinant or synthetic TLR5 agonist (e.g., a flagellin-based agent or variant thereof). In various embodiments, the patient is immunosenescent and/or immunocompromised. In some embodiments, the patient is a geriatric patient.

Some of the aspects and embodiments of this instant disclosure are based, at least in part, on the finding that TLR5 agonists (e.g., recombinant or synthetic flagellin and/or flagellin-based agents, such as entolimod), can be effective, for example, in improving and/or increasing vaccine efficacy in various patient populations, including, but not limited to, geriatric patients.

Without wishing to be bound by theory, the present invention contemplates that immune response to an antigen can be improved and/or enhanced by activation of natural innate immunity mechanism of response to infection with bacteria that have flagella—an organelle for active moving that is built with the protein named flagellin; presence of such bacteria in the body is recognized by a cell surface receptor named Toll-like receptor 5 (TLR5). Binding of a TLR5 agonist, e.g. a flagellin or flagellin-based agent (such as entolimod) to TLR5 triggers a physiological response leading to systemic mobilization of immune system accompanied with production of multiple bioactive factors (cytokines, chemokines, etc.) that have long-term effect on the organism manifested as a slowdown of frailty acquisition and improved health and quality of life of the treated organisms. Treatment with flagellin or its derivatives capable of activation of TLR5 can be projected as an approach to enhance and/or improve vaccine efficacy or to mitigate the effects of age-related immunosenescence.

### Immunosenescence

As described herein, immunosenescence refers to an age-dependent decrease in immunological competence resulting from a progressive deterioration of innate (e.g., neutrophils, macrophages and NK cells) and adaptive (dendritic cells and T-cells) immune responses.

In an aspect, the invention relates to methods for treating immunosenescence in a subject by administering to the subject an amount of TLR5 agonist (e.g., a flagellin-based agent), such as entolimod, effective to increase the immune response to an antigen (e.g., a vaccine antigen) so that protective antibody titers or T cell response to the antigen are achieved. In some embodiments, the antigen contemplated by the present invention is associated with and/or stimulates immunity against one or more of a cell having damaged DNA and a senescent cell.

The present invention contemplates, in some embodiments, methods of administering to the subject an amount of a TLR5 agonist (e.g., a flagellin-based agent), such as entolimod, for stimulation and mobilization of innate and adaptive immunity. For example, in some embodiments, the TLR5 agonist induces changes in the proportions and tissue distribution and activation of innate immunity cellular components (neutrophils, macrophages and NK cells) in solid tissues (i.e., liver, lungs, bladder), followed by the mobilization of adaptive immunity (dendritic and T-cells).

In some embodiments, the patient is affected with a chronic disease leading to an impaired immune function or immunosenescence such as a patient with diabetes including diabetes mellitus, a patient with chronic obstructive pulmonary disease (COPD) or a patient with sickle cell disease.

In some embodiments, immunosenescence is a decrease in immune function resulting in impaired immune response, e.g., to cancer, vaccination, infectious pathogens, among others. It involves both the host's capacity to respond to infections and the development of long-term immune memory, especially by vaccination. This immune deficiency is ubiquitous and found in both long- and short-lived species as a function of their age relative to life expectancy rather than chronological time. It is considered a major contributory factor to the increased frequency of morbidity and mortality among the elderly an/or geriatric. Immunosenescence is not a random deteriorative phenomenon, rather it appears to inversely repeat an evolutionary pattern and most of the parameters affected by immunosenescence appear to be under genetic control. Immunosenescence can also be envisaged as the result of the continuous challenge of the unavoidable exposure to a variety of antigens such as viruses and bacteria. Immunosenescence is a multifactorial condition leading to many pathologically significant health problems, e.g., in the aged population. Age-dependent biological changes such as depletion of hematopoietic stem cells, an increase in PD1+lymphocytes, a decline in the total number of phagocytes and NK cells and a decline in humoral immunity contribute to the onset of immunosenescence. In one aspect, immunosenescence can be measured in an individual by measuring telomere length in immune cells (See, e.g., U.S. Pat. No. 5,741,677). Immunosenescence can also be determined by documenting in an individual a lower than normal number of naïve CD4 and/or CD8 T cells, T cell repertoire, the number of PD1-expressing T cells, e.g., a lower than normal number of PD-1 negative T cells, or response to vaccination in a subject greater than or equal to 65 years of age.

In some embodiments, impaired immune response is a state in which a subject does not have an appropriate immune response, e.g., to cancer, vaccination, pathogen infection, among others. In some embodiments, a subject having an impaired immune response is predicted not to get protective antibody titer levels following prophylactic vaccination, or in which a subject does not have a decrease in disease burden after therapeutic vaccination. A subject can also have an impaired immune response if the subject is a member of a population known to have decreased immune function or that has a history of decreased immune function such as the elderly, subjects undergoing chemotherapy treatment, asplenic subjects, immunocompromised subjects, or subjects having HIV/AIDS. In some embodiments, a subject (e.g., an elderly subject) has an impaired immune response due to polypharmacy, or being administered many different drugs and/or therapeutics, that suppresses the subject's immune response. Methods described herein allow for the treatment of an impaired immune response by administration of an immune enhancing dose of a TLR5 agonist, e.g., a flagellin-based agent, such as entolimod.

In some embodiments, immunosenescence includes reduced immune response to infection with age and results from thymic involution in T-cell lineages, resulting in decreased T cell production and export (see e.g., Shimatani, K et al. (2009) PNAS 106 (37):15807-15812). In some embodiments, there is an increase in population of a bona fide age-dependent CD4+ T cell population defined by a constitutive expression of PD-1, which is induced only transiently on activation in regular T cells and, therefore, reduced immune response to infection (see e.g., Shimatani, K et al. (2009) PNAS 106 (37):15807-15812). In some embodiments, there is an increase in population of CD8+ T cell population defined by increased expression of PD-1 upon receptor-mediated activation of CD8+ T cells (see e.g., Nunes, C et al. (2012) Clinical Cancer Research 18(3):678-687). In some embodiments, immunosenescence comprises cellular senescence, in which a cell no longer divides. In some embodiments, age-related immunosenescence comprises decreased production of naive lymphocytes by hematopoietic stem cells (Chen, Science Signaling, ra75, 2009). Cellular senescence is correlated with the progressive shortening of telomeres that occurs with each cell division.

### TLR5 Agonists and Derivatives Thereof

Toll-like receptors (TLRs) play a central role in the initiation of cellular innate immune responses. They recognize pathogen-associated molecular patterns (PAMPs) that are expressed on infectious agents and mediate the production of cytokines necessary for the development of effective immunity. There are 10 TLR genes in humans and 12 in mice. In particular, Toll-like receptor 5 (TLR5) is a transmembrane protein that recognizes bacterial flagellin and is highly expressed in the intestinal mucosa. Vertebrate organisms recognize the presence of potentially pathogenic flagella-carrying bacteria via signaling activated by a highly specific interaction of flagellin with TLR5 that triggers a cascade of signal transduction events aimed at activation and mobilization of natural defense mechanisms of innate immunity. Activation of TLR5 by entolimod (CBLB502), a pharmacologically-useful flagellin derivative, was capable of protecting animals from lethal total body irradiation.

In some embodiments, a TLR5 agonist selectively activates or increases normal signal transduction through TLR5. In some embodiments of the present invention, a TLR5 agonist is recombinant or synthetic. In some embodiments, a TLR5 agonist has an EC50 of less than about 10⁻⁷M; or less than 10⁻⁸M; or less than 10⁻⁹M; or less than 10⁻¹⁰ M; or less than 10⁻¹⁰ M; or less than 10⁻¹¹M. In certain embodiments, a TLR5 agonist as provided herein has an EC50 of less than about 10⁻⁷M; or less than 10⁻⁸M; or less than 10⁻⁹M; or less than 10⁻¹⁰ M; or less than 10⁻¹⁰ M; or less than 10⁻¹¹M in the flagellin bioactivity assay using HEK-Blue™-hTLR5 cells (Invivogen) as described in Lu Y., et al., Biotechnol. Bioeng. 110, 2073-2085 (2013) and in Lu and Swartz, Sci Rep 6:18379 (2016) or a similar TLR5 bioactivity assay.

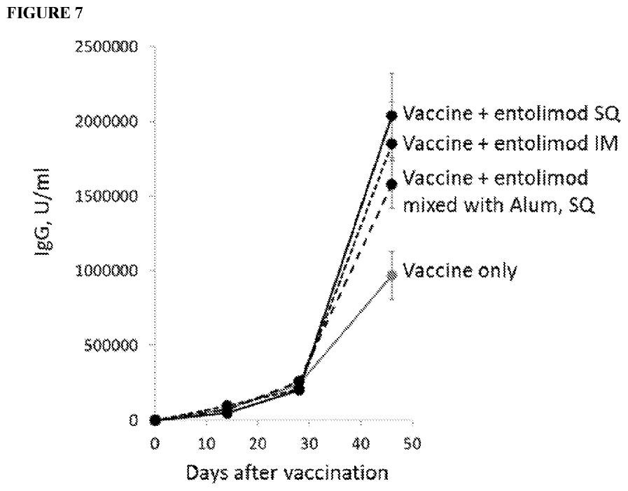

In some embodiments, a TLR5 agonist as provided herein is recombinant. In some embodiments, a TLR5 agonist as provided herein is synthetic. In some embodiments, a TLR5 agonist as provided herein is a flagellin-based agent. In some embodiments, a flagellin-based agent is contained in a variety of Gram-positive or Gram-negative bacterial species. The nucleotide and amino acid sequences of flagellin from 22 bacterial species are provided in FIG. 7 of United States Patent Publication No. 2003/0044429, which is hereby incorporated by reference in its entirety. Therefore, the sequence differences between species is included within the meaning of the term. In certain embodiments a flagellin-based agent in accordance with the present disclosure includes an amino acid sequence having at least 80% identity, or at least 85% identity, or at least 90% identity, or at least 95% identity, or at least 97% identity, or at least 98% identity, or at least 99% identity, or 100% identity with one or more of the flagellin from 22 bacterial species provided in FIG. 7 of United States Patent Publication No. 2003/0044429. The amino acid sequences of the conserved amino and carboxy terminus (important for TLR5 activity) from 21 species of bacteria are provided in FIG. 24A and 24B of U.S. Pat. No. 8,007,812, which is hereby incorporated by reference in its entirety.

In certain embodiments, a flagellin-based agent in accordance with the present disclosure includes a fragment of a flagellin protein or a flagellin-based agent. In some embodiments a flagellin based-agent or fragment thereof has activity as a TLR5 agonist. In various embodiments,

In some embodiments, the TLR5 agonist is a Salmonella flagellin protein, e.g. a recombinant or synthetic Salmonella flagellin protein. In some embodiments, the TLR5 agonist is a Salmonella dub/in flagellin protein, e.g. a recombinant or synthetic Salmonella dub/in flagellin protein. In various embodiments, the Salmonella dub/in flagellin protein has the amino acid sequence of SEQ ID NO: 27, as shown below:

(SEQ ID NO: 27)
MAQVINTNSLSLLTQNNLNKSQSSLSSAIERLSSGLRINSAKDDAAGQAI
ANRFTSNIKGLTQASRNANDGISIAQTTEGALNEINNNLQRVRELSVQAT
NGTNSDSDLKSIQDEIQQRLEEIDRVSNQTQFNGVKVLSQDNQMKIQVGA
NDGETITIDLQKIDVKSLGLDGFNVNGPKEATVGDLKSSFKNVTGYDTYA
AGADKYRVDINSGAVVTDAAAPDKVYVNAANGQLTTDDAENNTAVDLFKT
TKSTAGTAEAKAIAGAIKGGKEGDTFDYKGVTFTIDTKTGDDGNGKVSTT
INGEKVTLTVADIATGAADVNAATLQSSKNVYTSVVNGQFTFDDKTKNES
AKLSDLEANNAVKGESKITVNGAEYTANATGDKITLAGKTMFIDKTASGV
STLINEDAAAAKKSTANPLASIDSALSKVDAVRSSLGAIQNRFDSAITNL
GNTVTNLNSARSRIEDADYATEVSNMSKAQILQQAGTSVLAQANQVPQNV
LSLLR.

In some embodiments, the present invention contemplates use of a TLR5 agonist comprising a polypeptide having an amino acid sequence having at least about 80%, at least about 85%, at least about 87%, at least about 90%, at least about 93% at least about 95%, or at least about 96%, or at least about 97% or at least about 98%, or at least about 99%, or 100% sequence identity to SEQ ID NO: 27. In various embodiments, the polypeptide having an amino acid sequence does not comprise a His tag.

In some embodiments of the methods provided herein, the TLR5 agonist is entolimod (CBLB502). Entolimod (CBLB502) is a flagellin-related polypeptide (see, e.g., FIG. 7 of U.S. Patent Publication No. 2003/0044429, the contents of which are incorporated herein by reference in their entirety). As used herein, “entolimod” (aka “CBLB502”) refers to a polypeptide which has the sequence of SEQ ID NO: 1 of WIPO Patent Application WO/2016/109002 (hereby incorporated by reference in its entirety), as shown below:

(SEQ ID NO: 1)
MRGSHHHHHHGMASMTGGQQMGRDLYDDDDKDPMAQVINTNSLSLLTQNN
LNKSQSSLSSAIERLSSGLRINSAKDDAAGQAIANRFTSNIKGLTQASRN
ANDGISIAQTTEGALNEINNNLQRVRELSVQATNGTNSDSDLKSIQDEIQ
QRLEEIDRVSNQTQFNGVKVLSQDNQMKIQVGANDGETITIDLQKIDVKS
LGLDGFNVNSPGISGGGGGILDSMGTLINEDAAAAKKSTANPLASIDSAL
SKVDAVRSSLGAIQNRFDSAITNLGNTVTNLNSARSRIEDADYATEVSNM
SKAQILQQAGTSVLAQANQVPQNVLSLLR.

In some embodiments, the present invention contemplates use of a TLR5 agonist comprising a polypeptide having an amino acid sequence having at least about 80%, at least about 85%, at least about 87%, at least about 90%, at least about 93% at least about 95%, or at least about 96%, or at least about 97% or at least about 98%, or at least about 99%, or 100% sequence identity to SEQ ID NO: 1. In various embodiments, the polypeptide having an amino acid sequence does not comprise a His tag.

In some embodiments of the aspects and embodiments provided herein, the TLR5 agonist is a flagellin-based agent comprising a polypeptide having an amino acid sequence having at least 80% identity, or at least 85% identity, or at least 90% identity, or at least 95% identity, or at least 97% identity, or at least 98% identity, or at least 99% identity, or 100% identity with one or more of CBLB502-S33ML (SEQ ID NO: 35 of WO/2016/019034), CBLB502-485CT (CBLB533, SEQ ID NO: 71 of WO/2016/019034), CBLB502-S33MX (CBLB543, SEQ ID NO: 150 of WO/2016/019034), CBLB502-S33 (SEQ ID NO: 17 of WO/2016/019034), Mutant 33ML (SEQ ID NO: 42 of WO 2016/019034) of International Patent Application WO 2016/019034 (hereby incorporated by reference in its entirety), as shown below, respectively:

CBLB502-S33ML (SEQ ID NO: 35 of WO/2016/019034)
(SEQ ID NO: 2)
MSGLRINSAKDDAAGQAIANRFTSNIKGLTQASRNANDGISIAQTTEGAL
NEINNNLQRVRELSVQATNGTNSDSDLKSIQDEIQQRLEEIDRVSNQTQF
NGVKVLSQDNQMKIQVGANDGETITIDLQKIDVKSLGLDGFNVNSPGISG
GGGGILDSMGTLINEDAAAAKKSTANPLASIDSALSKVDAVRSSLGAIQN
RFDSAITNLGNTVTNLNSARSRIEDADYATEVSNMSKAQILQQAGTSVLA
QANQVPQNVLSLLVPRGSHHHHHHG;
CBLB502-485CT (CBLB533, SEQ ID NO: 71 of WO/2016/
019034)
(SEQ ID NO: 3)
MSGLRINSAKDDAAGQAIANRFTSNIKGLTQASRNANDGISIAQTTEGAL
NEINNNLQRVRELSVQATNGTNSDSDLKSIQDEIQQRLEEIDRVSNQTQF
NGVKVLSQDNQMKIQVGANDGETITIDLQKIDVKSLGLDGFNVNSPGSTA
NPLASIDSALSKVDAVRSSLGAIQNRFDSAITNLGNTVTNLNSARSRIED
ADYATEVSNMSKAQILQQAGLVPRGSHHHHHHG;
CBLB502-S33MX (CBLB543, SEQ ID NO: 150 of WO/2016/
019034)
(SEQ ID NO: 4)
MSGLRINSAKDDAAGQAIANRFTSNIKGLTQASRNAADGISIAQTTEGAL
NEINNNLQRVRELSVQATAGANADAALKAIQAEIQQRLEEIDRVSQQTQA
AAVKVLSQDNAMAIQVGANDGAAITIDLQKIDVKSLGLDGFNVNSPGSTA
NPLASIDSALSKVDAVRSSLGAIQNRFDSAITNLGNTVTNLNSARSRIED
ADYATEVSQMSKAQILQQAGTSVLAQANQVPQNVLSLLVPRGSHHHHHH
G;
CBLB502-S33 (SEQ ID NO: 17 of WO/2016/019034)
(SEQ ID NO: 5)
MRGSHHHHHHGMASMTGGQQMGRDLYDLVPRGSAKDPSGLRINSAKDDAA
GQAIANRFTSNIKGLTQASRNANDGISIAQTTEGALNEINNNLQRVRELS
VQATNGTNSDSDLKSIQDEIQQRLEEIDRVSNQTQFNGVKVLSQDNQMKI
QVGANDGETITIDLQKIDVKSLGLDGFNVNSPGISGGGGGILDSMGTLIN
EDAAAAKKSTANPLASIDSALSKVDAVRSSLGAIQNRFDSAITNLGNTVT
NLNSARSRIEDADYATEVSNMSKAQILQQAGTSVLAQANQVPQNVLSLL
R;
and
Mutant 33ML (SEQ ID NO: 42 of WO/2016/019034)
(SEQ ID NO: 6)
MSGLRINSAKDDAAGQAIANRFTSNIKGLTQASRNANDGISIAQTTEGAL
NEINNNLQRVRELSVQATNGTNSDSDLKSIQDEIQQRLEEIDRVSNQTQF
NGVKVLSQDNQMKIQVGANDGETITIDLQKIDVKSLGLDGFNVNSPGSTA
NPLASIDSALSKVDAVRSSLGAIQNRFDSAITNLGNTVTNLNSARSRIED
ADYATEVSNMSKAQILQQAGTSVLAQANQVPQNVLSLLVPRGSHHHHHH
G.

In some embodiments, the present invention contemplates use of a TLR5 agonist comprising a polypeptide having an amino acid sequence having at least about 80%, at least about 85%, at least about 87%, at least about 90%, at least about 93% at least about 95%, or at least about 96%, or at least about 97% or at least about 98%, or at least about 99%, or 100% sequence identity to one or more of SEQ ID NOs: 2-6. In various embodiments, the polypeptide having an amino acid sequence does not comprise a His tag.

In some embodiments of the aspects and embodiments provided herein, the TLR5 agonist is a flagellin-based agent comprising a polypeptide having an amino acid sequence having at least 80% identity, or at least 85% identity, or at least 90% identity, or at least 95% identity, or at least 97% identity, or at least 98% identity, or at least 99% identity or 100% identity with one or more of SEQ ID NOs: 243-252 of International Patent Application WO 2016/019134 (hereby incorporated by reference in its entirety), as shown below, respectively:

SEQ ID NO: 243 of WO 2016/019134
(SEQ ID NO: 7)
MGHHHHHHSGMEEFNMRINTNVAAMNTYSRLTAANTAKSNSLAKLSSGLR
INKAGDDAAGLAISEKMKSQIGGLTQAKRNAQDGISLVQTAEGALNETHS
ILERMRDLAVQGSNGTLTSSDRGSINKELKALHQELTRISNTTEFNTQKL
FSQTKQKSVTFTFQIGANAGQTLSVAITAMSGEALLVSTDAKFSLNAAGT
NAGAMIKSIDAAIAKVSDQRADLGAVQNRLEHTINNLTATNENLSDANSR
IRDVDMAEEMMTFTKSNILSQAATSMLAQANAMPNSVLNLLQG;
SEQ ID NO: 244 of WO 2016/019134
(SEQ ID NO: 8)
MGHHHHHHSGMRINHNISALNAWRNIDQTQYSMSKTLERLSSGLRINRAG
DDAAGLAISEKMRGQIKGLNMAIKNAQDAISLIQTAEGALTEVHSILQRM
RELAVQAASDTNTNVDREQIQKEIDQLREEIDRIARTTEFNTKKLLDGKL
EGFRSQVDAKVVTGGNINVQLGTVSSKAVEGTYVIEVGAAERAIMVVDAA
IHRVSTARAALGAIQNRLEHTISNLGVAAENLTAAESRIRDADMAKEMME
FTKQQILLQSSMAMLAQSNTLPQNVLQLMR;
SEQ ID NO: 245 of WO 2016/019134
(SEQ ID NO: 9)
MGHHHHHHSGLNMAIKNAQDAISLIQTAEGALTEVHSILQRMRELAVQAA
SDTNTNVDREQIQKEIDQLREEIDRIARTTEFNTKKLLDGKLEGFRSQVD
AKVVTGGNINVQLGTVSSKAVEGTYVIEVGAAERAIMVVDAAIHRVSTAR
AALGAIQNRLEHTISNLG;
SEQ ID NO: 246 of WO 2016/019134
(SEQ ID NO: 10)
MGHHHHHHSGMSLRINNNIEALNAWRALNSTSNALQKSMEKLSSGLRINR
AGDDAAGLAISEKLRAQIRGLNQAIRNAQDGISLIQTAEGGLSEIQNILQ
RMRELGVQAANGTLNNQDISAITTELNQLFNEIDRIAGATEFNTKNLLAV
STGLVVTLQVGANAGQVIAFTIDNAGTASLGLSSADLAINDNASASAFIS
KVDSALQKVSTYRANLGSIQNRLEHTIANLGIASENLSASESRIRDVDMA
AEMMNFTKNQILQQAGVAILAQANQAPQAVLQLLR;
SEQ ID NO: 247 of WO 2016/019134
(SEQ ID NO: 11)
MGHHHHHHSGLNQAIRNAQDGISLIQTAEGGLSEIQNILQRMRELGVQAA
NGTLNNQDISAITTELNQLFNEIDRIAGATEFNTKNLLAVSTGLVVTLQV
GANAGQVIAFTIDNAGTASLGLSSADLAINDNASASAFISKVDSALQKVS
TYRANLGSIQNRLEHTIANLG;
SEQ ID NO: 248 of WO 2016/019134
(SEQ ID NO: 12)
MGHHHHHHSGLNQAIRNAQDGISLIQTAEGGLSEIQNILQRMRELGVQAA
NGTLNNQDISAITTELNQLFNEIDRIAGATEFNTKNLLAAGTASLGLSSA
DLAINDNASASAFISKVDSALQKVSTYRANLGSIQNRLEHTIANLG;
SEQ ID NO: 249 of WO 2016/019134
(SEQ ID NO: 13)
MGHHHHHHSASAFISKVDSALQKVSTYRANLGSIQNRLEHTIANLGPDGL
NQAIRNAQDGISLIQTAEGGLSEIQNILQRMRELGVQAANGTLNNQDISA
ITTELNQLFNEIDRIA;
SEQ ID NO: 250 of WO 2016/019134
(SEQ ID NO: 14)
MGHHHHHHSNNQDISAITTELNQLFNEIDRIAGATGSGGLSEIQNILQRM
RELGVQAANGTLNGGSASAFISKVDSALQKVSTYRANLGSIQNRLEHTIA
NLG;
SEQ ID NO: 251 of WO 2016/019134
(SEQ ID NO: 15)
MGHHHHHHSGLAQASRNAQDAISIAQTAEGALDETQSILQRVRELGVQGA
NGTLTADDINALQAEVDQLIAEIDRIAGATEFNTQNLLDGSFTTKAFQVG
ANSGQNMTLTIGKMDTTTLGLSSADLAINDNAFANGAISTVDSALQKVSA
ERAKLGAIQNRLEHTIANLG;
and
SEQ ID NO: 252 of WO 2016/019134
(SEQ ID NO: 16)
MGHHHHHHSGLAQASRQAQDAISIAQTAEGALDETQSILQRVRELGVQGA
DGTLTADDIDALQAEVDQLIAEIDRIAGATEFATQKLLDGSFTTKAFQVG
AASGQDVTLTIGKVDTTTLGLSSADLAIDSAAFADGAISTVDSALQKVSA
ERAKLGAIQNRLEHTIAQLG.

In some embodiments, the present invention contemplates use of a TLR5 agonist comprising a polypeptide having an amino acid sequence having at least about 80%, at least about 85%, at least about 87%, at least about 90%, at least about 93% at least about 95%, or at least about 96%, or at least about 97% or at least about 98%, or at least about 99%, or 100% sequence identity to one or more of SEQ ID NOs: 7-16. In various embodiments, the polypeptide having an amino acid sequence does not comprise a His tag.

In some embodiments of the aspects and embodiments provided herein, the TLR5 agonist is a flagellin-based agent comprising a polypeptide having an amino acid sequence having at least 80% identity, or at least 85% identity, or at least 90% identity, or at least 95% identity, or at least 97% identity, or at least 98% identity, or at least 99% identity or 100% identity with one or more of SEQ ID NOs: 10, 12, 30, 32, 34, 36, 38, 40, 42, or 44 of International Patent Application WO 2006/069198 (hereby incorporated by reference in its entirety), as shown below, respectively:

SEQ ID NO: 10 of WO 2006/069198
(SEQ ID NO: 17)
MRGSHHHHHHGMASMTGGQQMGRDLYDDDDKDPMAQVINTNSLSLLTQNN
LNKSQSSLSSAIERLSSGLRINSAKDDAAGQAIANRFTSNIKGLTQASRN
ANDGISIAQTTEGALNEINNNLQRVRELSVQATNGTNSDSDLKSIQDEIQ
QRLEEIDRVSNQTQFNGVKVLSQDNQMKIQVGANDGETITIDLQKIDVKS
LGLDGFNVNSPGISGGGGGILDSMGTLINEDAAAAKKSTANPLASIDSAL
SKVDAVRSSLGAIQNRFDSAITNL;
SEQ ID NO: 12 of WO 2006/069198
(SEQ ID NO: 18)
MRGSHHHHHHGMASMTGGQQMGRDLYDDDDKDPFTSNIKGLTQASRNAND
GISIAQTTEGALNEINNNLQRVRELSVQATNGTNSDSDLKSIQDEIQQRL
EEIDRVSNQTQFNGVKVLSQDNQMKIQVGANDGETITIDLQKIDVKSLGL
DGFNVNSPGISGGGGGILDSMGTLINEDAAAAKKSTANPLASIDSALSKV
DAVRSSLGAIQNRFDSAITNLGNIVINLNSARSRIEDADYATEVSNMSKA
QILQQAGTSVLAQANQVPQNVLSLLR;
SEQ ID NO: 30 of WO 2006/069198
(SEQ ID NO: 19)
MRGSHHHHHHGMASMTGGQQMGRDLYDDDDKDPMAQVINTNSLSLLTQNN
LNKSQSSLSSAIERLSSGLRINSAKDDAAGQAIANRFTSNIKGLTQASRN
ANDGISIAQTTEGALNEINNNLQRVRELSVQATNGTNSDSDLKSIQDEIQ
QRLEEIDRVSNQTQFNGVKVLSQDNQMKIQVGANDGETITIDLQKIDVKS
LGLIPGISGGGGGILDSMGTLINEDAAAAKKSTANPLASIDSALSKVDAV
RSSLGAIQNRFDSAITNLGNIVINLNSARSRIEDADYATEVSNMSKAQIL
QQAGTSVLAQANQVPQNVLSLLR;
SEQ ID NO: 32 of WO 2006/069198
(SEQ ID NO: 20)
MRGSHHHHHHGMASMTGGQQMGRDLYDDDDKDPFTSNIKGLTQASRNAND
GISIAQTTEGALNEINNNLQRVRELSVQATNGTNSDSDLKSIQDEIQQRL
EEIDRVSNQTQFNGVKVLSQDNQMKIQVGANDGETITIDLQKIDVKSLGL
IPGISGGGGGILDSMGTLINEDAAAAKKSTANPLASIDSALSKVDAVRSS
LGAIQNRFDSAITNLGNIVINLNSARSRIEDADYATEVSNMSKAQILQQA
GTSVLAQANQVPQNVLSLLR;
SEQ ID NO: 34 of WO 2006/069198
(SEQ ID NO: 21)
MRGSHHHHHHGMASMTGGQQMGRDLYDDDDKDPMAQVINTNSLSLLTQNN
LNKSQSSLSSAIERLSSGLRINSAKDDAAGQAIANRFTSNIKGLTQASRN
ANDGISIAQTTEGALNEINNNLQRVRELSVQATNGTNSDSDLKSIQDEIQ
QRLEEIDRVSNQTQFNGVKVLSQDNQMKIQVGANDGETITIDLQKIIPGI
SGGGGGILDSMGTLINEDAAAAKKSTANPLASIDSALSKVDAVRSSLGAI
QNRFDSAITNLGNIVINLNSARSRIEDADYATEVSNMSKAQILQQAGTSV
LAQANQVPQNVLSLLR;
SEQ ID NO: 36 of WO 2006/069198
(SEQ ID NO: 22)
MRGSHHHHHHGMASMTGGQQMGRDLYDDDDKDPFTSNIKGLTQASRNAND
GISIAQTTEGALNEINNNLQRVRELSVQATNGTNSDSDLKSIQDEIQQRL
EEIDRVSNQTQFNGVKVLSQDNQMKIQVGANDGETITIDLQKIIPGISGG
GGGILDSMGTLINEDAAAAKKSTANPLASIDSALSKVDAVRSSLGAIQNR
FDSAITNLGNIVINLNSARSRIEDADYATEVSNMSKAQILQQAGTSVLAQ
ANQVPQNVLSLLR
SEQ ID NO: 38 of WO 2006/069198
(SEQ ID NO: 23)
MRGSHHHHHHGMASMTGGQQMGRDLYDDDDKDPMAQVINTNSLSLLTQNN
LNKSQSSLSSAIERLSSGLRINSAKDDAAGQAIANRFTSNIKGLTQASRN
ANDGISIAQTTEGALNEINNNLQRVRELSVQATNGTNSDSDLKSIQDEIQ
QRLEEIDRVSNQTQFNGVKVLSQDNQMKIQVGANDGETITIDLQKIDVKS
LGLIPGISGGGGGILDSMGTLINEDAAAAKKSTANPLASIDSALSKVDAV
RSSLGAIQNRFDSAITNL;
SEQ ID NO: 40 of WO 2006/069198
(SEQ ID NO: 24)
MRGSHHHHHHGMASMTGGQQMGRDLYDDDDKDPMAQVINTNSLSLLTQNN
LNKSQSSLSSAIERLSSGLRINSAKDDAAGQAIANRFTSNIKGLTQASRN
ANDGISIAQTTEGALNEINNNLQRVRELSVQATNGTNSDSDLKSIQDEIQ
QRLEEIDRVSNQTQFNGVKVLSQDNQMKIQVGANDGETITIDLQKIIPGI
SGGGGGILDSMGTLINEDAAAAKKSTANPLASIDSALSKVDAVRSSLGAI
QNRFDSAITNL;
SEQ ID NO: 42 of WO 2006/069198
(SEQ ID NO: 25)
MRGSHHHHHHGMASMTGGQQMGRDLYDDDDKDPMAQVINTNSLSLLTQNN
LNKSQSSLSSAIERLSSGLRINSAKDDAAGQAIANRFTSNIKGLTQASRN
ANDGISIAQTTEGALNEINNNLQRVRELSVQATNGTNSDSDLKSIQDEIQ
QRLEEIDRVSNQIPGISGGGGGILDSMGTLINEDAAAAKKSTANPLASID
SALSKVDAVRSSLGAIQNRFDSAITNLGNTVTNLNSARSRIEDADYATEV
SNMSKAQILQQAGTSVLAQANQVPQNVLSLLR;
and
SEQ ID NO: 44 of WO 2006/069198
(SEQ ID NO: 26)
MRGSHHHHHHGMASMTGGQQMGRDLYDDDDKDPFTSNIKGLTQASRNAND
GISIAQTTEGALNEINNNLQRVRELSVQATNGTNSDSDLKSIQDEIQQRL
EEIDRVSNQIPGISGGGGGILDSMGTLINEDAAAAKKSTANPLASIDSAL
SKVDAVRSSLGAIQNRFDSAITNLGNTVTNLNSARSRIEDADYATEVSNM
SKAQILQQAGTSVLAQANQVPQNVLSLLR.

In some embodiments, the present invention contemplates use of a TLR5 agonist comprising a polypeptide having an amino acid sequence having at least about 80%, at least about 85%, at least about 87%, at least about 90%, at least about 93% at least about 95%, or at least about 96%, or at least about 97% or at least about 98%, or at least about 99%, or 100% sequence identity to one or more of SEQ ID NOs: 17-26. In various embodiments, the polypeptide having an amino acid sequence does not comprise a His tag.

In some embodiments of the aspects and embodiments provided herein, the TLR5 agonist is a flagellin-based agent comprising a polypeptide having an amino acid sequence having at least 80% identity, or at least 85% identity, or at least 90% identity, or at least 95% identity, or at least 97% identity, or at least 98% identity, or at least 99% identity or 100% identity with SEQ ID NO: 28, as shown below:

(SEQ ID NO: 28)
MSGLRINSAKDDAAGQAAANRATSNIKGLTQASRNAADGISIAQTTEGAL
NEINNNLQRVRELSVQATAGANADAALKAIQAEIQQRLEEIDRVSQQTQA
AAVKVLSQDNAMAIQVGANDGAAITIDLQKIDVKSLGLDGFNVNSPGSTA
NPLASIDSALSKVDAVRSSLGAIQNRFDSAITNLGNTVTNLNSARSRIED
ADYATEVSQMSKAQILDQAGTSTLAQLVPRGSHHHHHHG.

In some embodiments, the present invention contemplates use of a TLR5 agonist comprising a polypeptide having an amino acid sequence having at least about 80%, at least about 85%, at least about 87%, at least about 90%, at least about 93% at least about 95%, or at least about 96%, or at least about 97% or at least about 98%, or at least about 99%, or 100% sequence identity to SEQ ID NO: 28. In various embodiments, the polypeptide having an amino acid sequence does not comprise a His tag.

According to the present invention, in some embodiments, a pathogenic protein antigen is not fused to a TLR5 agonist. Examples of the pathogenic protein antigen that in some embodiments would not be fused to a TLR5 agonist and/or flagellin based agent as described herein include an α-helix domain of surface protein A (PspA) and pneumococcal surface protein A (PsaA) of Streptococcus pneumonia; subunit hemagglutinin (HA) and neuraminidase (NA) of influenza virus; and spike (S) protein of severe acute respiratory syndrome virus (SARS virus), and the like.

### Antigens and Vaccines

In some embodiments, the present invention provides methods for improving and/or increasing vaccine efficacy in a patient, as measured by a reduction of vaccine dosage, relative to the vaccine dosage of a patient that was not administered the recombinant or synthetic TLR5 agonist. In some embodiments, the present invention provides methods for improving and/or increasing vaccine efficacy in a patient, as measured by a reduction of the frequency of vaccine dosing, relative to the frequency of vaccine dosing of a patient that was not administered the recombinant or synthetic TLR5 agonist.

In various embodiments, the antigens selected for the methods and compositions of the invention are not a limitation on this invention. The antigen may be, without limitation, a whole cell, a virus, a protein, a protein subunit or fragment. In further embodiments, the antigen which stimulates an immune response against a disorder is a constituent of an infectious agent selected from a live and attenuated, killed, inactivated, and toxoid infectious agent. In some embodiments, the antigen is associated with and/or stimulates an immune response against a tumor cell, a cell with damaged DNA, or a senescent cell.

Examples of viral antigens which may be enhanced by administration with a TLR5 agonist (e.g., a flagellin-based agent), include, without limitation, those derived from and/or useful in treatment or prevention of HIV, meningitis and encephalitis-causing viruses, Hepatitis A, Hepatitis B, Hepatitis C, rabies virus, polio virus, influenza virus, measles virus, mumps virus, rubella, pertussis, papilloma virus, yellow fever virus, respiratory syncytial virus, parvovirus, chikungunya virus, haemorrhagic fever viruses, and Herpes viruses, particularly, varicella, cytomegalovirus and Epstein-Barr virus.

Examples of bacterial and mycobacterial antigens include those derived from and/or useful against meningococcus, Haemophilus, pneumococcus, Staphylococcus, leprosy and tuberculosis, among others.

In particular, a TLR5 agonist (e.g., a flagellin-based agent), such as, for example, entolimod, can be used in combination with a vaccine against a viral or pathogenic agent, such as an influenza vaccine, pneumococcal vaccine, or HIV vaccine. More specifically, a TLR5 agonist can be used as described herein to enhance the immune response to a vaccine for any influenza strain, such as H1N1, H2N3, and B influenza subtypes.

In some embodiments, the present invention contemplates the use of antigens of one or more approved vaccines may be the antigens of the present invention. In some embodiments, the approved vaccines include: Adenovirus; Anthrax (Biothrax); BCG (Tice); DT (Sanofi); DTaP (Daptacel); DTaP (Infanrix); DTaP-HepB-IPV (Pediarix); DTaP-IPV (Kinrix); DTaP-IPV/Hib (Pentacel); Hib (ActHIB); Hib (Hiberix); Hib (PedvaxHlB); Hib/Hep B (Comvax); Hib/Mening. CY (MenHibrix); Hep A (Havrix); Hep A (Havrix); Hep B (Engerix-B); Hep B (Recombivax); Hep A/Hep B (Twinrix); Human Papillomavirus (HPV) (Cerverix); Human Papillomavirus (HPV) (Gardasil); Influenza (Afluria); Influenza (Agriflu); Influenza (Fluarix); Influenza (Flublok); Influenza (Flucelvax); Influenza (Fluvirin); Influenza (Flulaval); Influenza (Fluzone: Standard, High-Dose, & Intradermal); Influenza (FluMist); Japanese Encephalitis (Ixiaro); Meningococcal (MCV4-Menactra); Meningococcal (MCV4-Menveo); Meningococcal (MPSV4-Menomune); MMR (MMR-II); MMRV (ProQuad); Pneumococcal (PCV13—Prevnar 13); Pneumococcal (PPSV-23—Pneumovax); Polio (IPV—Ipol); Rabies (Imovax); Rabies (RabAvert); Rotavirus (RotaTeq); Rotavirus (Rotarix); Smallpox (Vaccinia—ACAM2000); Td (Decavac); Td (Tenivac); Td (Mass Biologics); Tdap (Adacel); Tdap (Boostrix); Typhoid (inactivated—Typhim Vi); Typhoid (oral—Ty21a); Varicella (Varivax); Yellow Fever (YF-Vax); and Zoster (Shingles—Zostavax).

In some embodiments, the present invention contemplates the use of antigens of one or more approved vaccines as the antigens of the present invention. Illustrative vaccines include, by way of example, subunit vaccine and inactivated or “killed” vaccine (e.g. Infanrix-IPV/Hib (Bordetella pertussis), Infanrix-IPV/Hib (Haemophilus influenzae), Infanrix-IPV/Hib (Poliovirus), Infanrix-IPV/Hib (Clostridium tetani), Infanrix-IPV/Hib (Corynebacterium diphtheriae), Infanrix-hexa (Bordetella pertussis), Infanrix-hexa (Haemophilus influenzae), Infanrix-hexa (Poliovirus), Infanrix-hexa (Hepatitis B virus), Infanrix-hexa (Clostridium tetani), Infanrix-hexa (Corynebacterium diphtheriae), Infanrix-IPV (Bordetella pertussis), Infanrix-IPV (Poliovirus), Infanrix-IPV (Clostridium tetani), Infanrix-IPV (Corynebacterium diphtheriae), Infanrix/Hib (Corynebacterium diphtheriae), Pediarix (Clostridium tetani), Pediarix (Poliovirus), Pediarix (Hepatitis B virus), ViVaxim (Salmonella spp.), ViVaxim (Hepatitis A virus); subunit vaccines (e.g. SCVMB (Neisseria meningitidis), B. pertussis CyaA protein vaccine (Bordetella pertussis), B. pertussis PTx protein vaccine (Bordetella pertussis), Cancer VEGFA protein vaccine (Cancer), E. coli vaccine using intimin polypeptide (Escherichia coli), Engerix-B (Hepatitis B virus), H. pylori VacA protein vaccine (Helicobacter pylori), HC of type C and D (Clostridium botulinum), Infanrix/Hib (Bordetella pertussis), Infanrix/Hib (Haemophilus influenzae), Infanrix/Hib (Clostridium tetani), M. gallisepticum TM-1 Protein Subunit Vaccine (Mycoplasma gallisepticum), MDA-modified human apo B-100 peptide Vaccine (Atherosclerosis), MSP3-LSP with aluminium hydroxide (Plasmodium spp.), Mumps HN Protein Subunit Vaccine (Mumps virus), N. miningitidis TBP2 Protein Vaccine (Neisseria meningitidis), P. aeruginosa OprI Protein Vaccine (Pseudomonas aeruginosa), P. falciparum Subunit SE36 Protein Vaccine (Plasmodium spp.), Phleum pratense Allergy Ph1 p 12 Subunit Vaccine (Allergy), Recombivax HB (Hepatitis B virus), S. pneumoniae ClpP protein Vaccine (Streptococcus pneumoniae); toxoid vaccine (e.g. BoNT/F(Hc) (Clostridium botulinum), DAPTACEL (Corynebacterium diphtheriae), Infanrix (Bordetella pertussis), Infanrix (Clostridium tetani), KINRIX (Clostridium tetani), PBT (Clostridium botulinum), Pediarix (Bordetella pertussis), inactivated or “killed” vaccines (e.g. Avaxim (Hepatitis A virus), Avaxim-Pediatric (Hepatitis A virus), FSME-IMMUN (Tick-borne Encephalitis Virus (TBEV)), Infanrix (Corynebacterium diphtheriae), Ixiaro (Japanese encephalitis virus), KINRIX (Corynebacterium diphtheriae), and Pediarix (Corynebacterium diphtheriae)); and conjugate vaccines (e.g., Arabinomannan-tetanus toxoid conjugate (Mycobacterium tuberculosis)), CCPS-P64kR (Neisseria meningitidis), COMVAX (Haemophilus influenzae), Menjugate (Neisseria meningitidis), Neisvac-C (Neisseria meningitidis), and PedvaxHlB (Haemophilus influenza)).

In various embodiments, the present invention contemplates that one or more cancer vaccines and/or the antigens of one or more cancer vaccines may be the antigens of the present invention. Illustrative cancer vaccines include therapeutic and preventative vaccines. For instance, cancer vaccines include ONCOPHAGE (ANTIGENICS INC., approved in Russia in 2008 for kidney cancer), APC8015/Sipuleucel-T/PROVENGE (DENDREON, for, e.g. metastatic hormone-refractory prostate cancer), CANCERVAX (CANVAXIN), GENITOPE CORP (MYVAX personalized immunotherapy), and FAVRILLE INC (FAVID), preventive vaccines which attack the cancer-causing viruses human papillomavirus (e.g. CERVARIX (GSK) and GARDASIL (MERCK)), hepatitis A virus (e.g. CERVARIX (GSK) and GARDASIL (MERCK)), and hepatitis B virus (e.g. RECOMBIVAX HB (MERCK), ENGERIX-B (GSK), ELOVAC B (HUMAN BIOLOGICALS INSTITUTE), GENEVAC B (SERUM INSTITUTE), SHANVAC B, etc.

### Pathogenic Infections

In another aspect, the methods provided herein can be used to treat infection by a pathogen in a subject. In some embodiments, the pathogen is a bacterial pathogen, e.g., a bacterial pathogen selected from Meningococcus, Haemophilus, Pneumococcus, Staphylococcus, Streptococcus, Neisseria, Moraxella, Escherichia coli, Klebsiella, Pseudomonas, Enterobacter, Proteus, Serratia, Legionella, Salmonella, Shigella, Acinetobacer, Listeria, Chlamydia, Mycobacterium, among others.

In some embodiments, the pathogen is a viral pathogen, e.g., a viral pathogen e.g. HIV, meningitis causing viruses, encephalitis causing viruses, Hepatitis A, Hepatitis B, Hepatitis C, rabies virus, polio virus, influenza virus, parainfluenza virus, adenovirus, rhinovirus, measles virus, mumps virus, rubella, pertussis, papilloma virus, yellow fever virus, respiratory syncytial virus, parvovirus, Norwalk virus, chikungunya virus, haemorrhagic fever viruses, dengue virus, and Herpes viruses, e.g., varicella, cytomegalovirus and Epstein-Barr virus. In some embodiments, the infection is a viral infection, such as a chronic viral infection. In some embodiments, a chronic viral infection is selected from Hepatitis A, Hepatitis B, Hepatitis C, Epstein Barr Virus, HIV, Cytomegalovirus, Herpes Simplex Virus 1, Herpes Simplex Virus 2, Human Papillomavirus, Adenovirus, and Kaposi's Sarcoma-Associated Herpesvirus. In some embodiments, a chronic viral infection comprises HIV.

For example, Lichterfeld and colleagues observed that HIV-specific CD8+ T-cells showed reduced telomere length and an increase in telomere length and telomerase activity upon inhibition of PD-1 (see e.g., Lichterfeld, M et al. (2008) Blood 112(9):3679-3687). In another example, PD-1 was significantly upregulated in hepatitis C (HVC)-specific CD8+ cytotoxic T lymphocytes (see e.g., Golden-Mason, L (2007) J. Virol. 81(17): 9249-9258).

In some embodiments, a viral infection comprises a viral acute lower respiratory tract infection. In some embodiments viral acute lower respiratory tract infection is caused by a rhinovirus, coronavirus, influenza virus, respiratory syncytial virus (RSV), adenovirus, and/or parainfluenza. In some embodiments, a viral acute lower respiratory tract infection is pnemonia. In some embodiments, a viral acute lower respiratory tract infection includes a lung abscess. In some embodiments, a viral acute lower respiratory tract infection includes bronchitis.

In some embodiments, the pathogen is a parasitic pathogen, e.g., Toxoplasma, Leishmania and malaria, T. cruzii, Helminth, e.g., Schistosoma.

In some embodiments, the pathogen is a yeast or fungal pathogen, e.g., Candida, Cryptococcus or Coccidioides.

### Cancer

The methods described herein can be used with any cancer. In an embodiment, the cancer comprises a solid tumor. In an embodiment, the cancer is a hematological cancer. The cancer can be a carcinoma, a sarcoma, a myeloma, a leukemia, a lymphoma or a mixed type. In some embodiments, the antigen contemplated by the present invention is associated with and/or stimulates immunity against a tumor cell.

Cancer vaccines typically include an antigen expressed on and isolated from a cancer cell or a cancer cell transfected with, and capable of expressing, a selected antigen. For example, any purified tumor antigen may be administered with a a TLR5 agonist (e.g., a flagellin-based agent), such as entolimod, as described for pathogenic vaccines. Identification of relevant cancer antigens will permit the development of such vaccines. Alternatively, other cancer therapeutics are designed using an antigen normally not expressed on a cancer cell. For example, a selected antigen may be transfected into the cancer cell and the transfected cell itself, expressing the antigen, is used as the vaccine or therapeutic.

In some embodiments, the cancer is associated with elevated percentages of PD1+ T cells in the subject. In certain embodiments, the cancer is a cancer that generally responds to PD-1 targeted drugs, such as melanoma. In certain embodiments, the cancer is a cancer that generally responds to T-cell directed immunotherapies, such as renal cell carcinoma. In an embodiment the cancer is one in which can be treated by increasing the ratio of PD-1 negative to PD-1 positive T cells.

Examples of cancers that can be treated with methods disclosed herein include bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers.

Examples of solid tumors that can be treated with methods disclosed herein include malignancies, e.g., sarcomas, adenocarcinomas, and carcinomas, of the various organ systems, such as those affecting liver, lung, breast, lymphoid, gastrointestinal (e.g., colon), genitourinary tract (e.g., renal, urothelial cells), prostate and pharynx. Adenocarcinomas include malignancies such as most colon cancers, rectal cancer, renal-cell carcinoma, liver cancer, non-small cell carcinoma of the lung, cancer of the small intestine and cancer of the esophagus. In one embodiment, the cancer is a melanoma, e.g., an advanced stage melanoma. Metastatic lesions of the aforementioned cancers can also be treated or prevented using the methods and compositions of the invention.

Methods described herein can be used to treat any of the following cancers:

Digestive/gastrointestinal cancers such as anal cancer; bile duct cancer; extrahepatic bile duct cancer; appendix cancer; carcinoid tumor, gastrointestinal cancer; colon cancer; colorectal cancer including childhood colorectal cancer; esophageal cancer including childhood esophageal cancer; gallbladder cancer; gastric (stomach) cancer including childhood gastric (stomach) cancer; hepatocellular (liver) cancer including adult (primary) hepatocellular (liver) cancer and childhood (primary) hepatocellular (liver) cancer; pancreatic cancer including childhood pancreatic cancer; sarcoma, rhabdomyosarcoma; islet cell pancreatic cancer; rectal cancer; and small intestine cancer;

Endocrine cancers such as islet cell carcinoma (endocrine pancreas); adrenocortical carcinoma including childhood adrenocortical carcinoma; gastrointestinal carcinoid tumor; parathyroid cancer; pheochromocytoma; pituitary tumor; thyroid cancer including childhood thyroid cancer; childhood multiple endocrine neoplasia syndrome; and childhood carcinoid tumor;

Eye cancers such as intraocular melanoma; and retinoblastoma;

Musculoskeletal cancers such as Ewing's family of tumors; osteosarcoma/malignant fibrous histiocytoma of the bone; childhood rhabdomyosarcoma; soft tissue sarcoma including adult and childhood soft tissue sarcoma; clear cell sarcoma of tendon sheaths; and uterine sarcoma;

Breast cancer such as breast cancer including childhood and male breast cancer and pregnancy;

Neurologic cancers such as childhood brain stem glioma; brain tumor; childhood cerebellar astrocytoma; childhood cerebral astrocytoma/malignant glioma; childhood ependymoma; childhood medulloblastoma; childhood pineal and supratentorial primitive neuroectodermal tumors; childhood visual pathway and hypothalamic glioma; other childhood brain cancers; adrenocortical carcinoma; central nervous system lymphoma, primary; childhood cerebellar astrocytoma; neuroblastoma; craniopharyngioma; spinal cord tumors; central nervous system atypical teratoid/rhabdoid tumor; central nervous system embryonal tumors; and childhood supratentorial primitive neuroectodermal tumors and pituitary tumor;

Genitourinary cancers such as bladder cancer including childhood bladder cancer; renal cell (kidney) cancer; ovarian cancer including childhood ovarian cancer; ovarian epithelial cancer; ovarian low malignant potential tumor; penile cancer; prostate cancer; renal cell cancer including childhood renal cell cancer; renal pelvis and ureter, transitional cell cancer; testicular cancer; urethral cancer; vaginal cancer; vulvar cancer; cervical cancer; Wilms tumor and other childhood kidney tumors; endometrial cancer; and gestational trophoblastic tumor;

Germ cell cancers such as childhood extracranial germ cell tumor; extragonadal germ cell tumor; ovarian germ cell tumor; and testicular cancer;

Head and neck cancers such as lip and oral cavity cancer; oral cancer including childhood oral cancer; hypopharyngeal cancer; laryngeal cancer including childhood laryngeal cancer; metastatic squamous neck cancer with occult primary; mouth cancer; nasal cavity and paranasal sinus cancer; nasopharyngeal cancer including childhood nasopharyngeal cancer; oropharyngeal cancer; parathyroid cancer; pharyngeal cancer; salivary gland cancer including childhood salivary gland cancer; throat cancer; and thyroid cancer;

Lung cancer such as non-small cell lung cancer; and small cell lung cancer;

Respiratory cancers such as malignant mesothelioma, adult; malignant mesothelioma, childhood; malignant thymoma; childhood thymoma; thymic carcinoma; bronchial adenomas/carcinoids including childhood bronchial adenomas/carcinoids; pleuropulmonary blastoma; non-small cell lung cancer; and small cell lung cancer;

Skin cancers such as Kaposi's sarcoma; Merkel cell carcinoma; melanoma; and childhood skin cancer;

AIDS-related malignancies;

Other childhood cancers, unusual cancers of childhood and cancers of unknown primary site; and metastases of the aforementioned cancers can also be treated or prevented in accordance with the methods described herein.

Methods described herein can be used to treat a hematological cancer or malignancy or precancerous condition, e.g., a leukemia or a lymphoma. The cancer can be one associated with expression of a cancer-associated antigen as described herein. Hematological cancers and malignancies include, one or more acute leukemias including, e.g., B-cell acute Lymphoid Leukemia (“BALL”), T-cell acute Lymphoid Leukemia (“TALL”), acute lymphoid leukemia (or acute lymphoblastic leukemia) (ALL), including adult and childhood acute lymphoid leukemia; acute myeloid leukemia, including adult and childhood acute myeloid leukemia; one or more chronic leukemias, e.g., chronic myelogenous leukemia (CIVIL), Chronic Lymphoid Leukemia (or chronic lymphocytic leukemia) (CLL). Additional cancers or hematologic conditions that can be treated with methods disclosed herein include, e.g., AIDS-related lymphoma, B cell prolymphocytic leukemia, blastic plasmacytoid dendritic cell neoplasm, Burkitt's lymphoma, chronic myeloproliferative disorders; cutaneous T-cell lymphoma, diffuse large B cell lymphoma, Follicular lymphoma, Hairy cell leukemia, Hodgkin's lymphoma (including adult and childhood Hogkin's lymphoma and Hodgkin's lymphoma during pregnancy), small cell- or a large cell-follicular lymphoma, malignant lymphoproliferative conditions, MALT lymphoma, mantle cell lymphoma, Marginal zone lymphoma, multiple myeloma, multiple myeloma/plasma cell neoplasm, myelodysplasia and myelodysplastic syndrome, myelodysplastic/myeloproliferative disorders, mycosis fungoides, non-Hodgkin's lymphoma (including adult and childhood non-Hodgkin's lymphoma and non-Hodkin's lymphoma during pregnancy), plasmablastic lymphoma, plasmacytoid dendritic cell neoplasm, Sezary syndrome, Waldenstrom macroglobulinemia, primary central system lymphoma, and “preleukemia” which are a diverse collection of hematological conditions united by ineffective production (or dysplasia) of myeloid blood cells, and the like. Further, a disease associated with a cancer-associated antigen as described herein expression includes, but is not limited to, e.g., atypical and/or non-classical cancers, malignancies, precancerous conditions or proliferative diseases associated with expression of a cancer-associated antigen, as described herein.

### Vaccine Efficacy

In some embodiments, the present invention provides methods for improving and/or increasing vaccine efficacy in a patient, as measured by an increase in the patient's innate and/or adaptive immune responses. In some embodiments, methods and compositions of the present invention for for improving and/or increasing vaccine efficacy in a patient include maintaining and/or increasing the patient's T cell populations (e.g., CD4+ and/or CD8+ T cell populations). In further embodiments, methods of the present invention provide for mitigation of age-related immunosenescence as measured by an increase or restoration of a patient's antigen-specific antibody titers (e.g., IgG, IgM and IgA).

In some embodiments, the present invention provides methods for improving and/or increasing vaccine efficacy in a patient, as measured by higher titer levels of antigen-specific antibodies as compared to titer levels of antigen-specific antibodies in patients that were not administered the TLR5 agonist. In some embodiments, the present invention provides methods for improving and/or increasing vaccine efficacy in a patient, as measured by an increase in the patient's innate immune response, as compared to the innate immune response of a patient that was not administered the TLR5 agonist. In further embodiments, the present invention provides methods for improving and/or increasing vaccine efficacy in a patient, as measured by an increase in the patient's adaptive immune response, as compared to the adaptive immune response of a patient that was not administered the TLR5 agonist. In some embodiments, the patient's innate immune response and adaptive immune response are increased, as compared to the innate and adaptive immune responses of a patient that was not administered the TLR5 agonist.

In some embodiments, the present invention provides methods for improving and/or increasing vaccine efficacy in a patient, as measured by an increase and/or restoration of the patient's T cell population(s), as compared to the T cell populations of a patient that was not administered the TLR5 agonist. For example, in further embodiments, the patient's T cells, including T cells selected from one or more of CD4+ effector T cells, CD8+ effector T cells, CD4+ memory T cells, CD8+ memory T cells, CD4+ central memory T cells, CD8+ central memory T cells, natural killer T cells, CD4+ helper cells (including, without limitation Th1, Th2, and Th17), and CD8+ cytotoxic cells, are increased and/or restored, as compared to the T cell populations of a patient that was not administered the TLR5 agonist.

More specifically, in some embodiments, the present invention provides methods for improving and/or increasing vaccine efficacy in a patient, as measured by an increase and/or restoration of the patient's T cell subsets. In some embodiments, the T cells are T helper cells (e.g., Tₕ cells). In further embodiments, T helper cells secrete cytokines that attract one or more of macrophages, neutrophils, other lymphocytes, and other cytokines to further direct these cells. In some embodiments, CD4+ T helper cells are one of several subsets, including, Th1, Th2, Th17, Th9, and Tfh, with each subset having a different function.

In some embodiments, T cells are cytotoxic cells that optionally produce IL-2 and IFNγ cytokines. In further embodiments, these T cells are cytotoxic CD8+ T cells (also known as Tc cells or T-killer cells).

In some embodiments, memory T cells elicited by methods of the present invention are long-lived and can expand to large numbers of effector T cells when re-exposed to their cognate antigen. In some embodiments, memory T cells provide a patient's immune system with memory against previously encountered pathogens. In further embodiments, memory T cell populations include, but are not limited to, tissue-resident memory T (Trm) cells, stem memory TSCM cells, and virtual memory T cells. In some embodiments, memory T cells are classified as CD4+ or CD8+ and express CD45RO. In some embodiments, memory T cells are further differentiated into various subsets. For example, in some embodiments, memory T cell subsets include: Central memory T cells (T_{CM} cells), which can express CD45RO, C-C chemokine receptor type 7 (CCR7), L-selectin (CD62L), and CD44; Effector memory T cells (T_{EM} cells and T_{EMRA} cells), which express CD45RO and CD44 but lack expression of CCR7 and CD62L; Tissue resident memory T cells (T_{RM}), which is associated with the integrin αeβ7; and Virtual memory T cells.

In some embodiments, the present invention provides methods for improving and/or increasing vaccine efficacy in a patient, as measured by a reduction of vaccine dosage, relative to the vaccine dosage of a patient that was not administered the recombinant or synthetic TLR5 agonist. In some embodiments, the present invention provides methods for improving and/or increasing vaccine efficacy in a patient, as measured by a reduction of the frequency of vaccine dosing, relative to the frequency of vaccine dosing of a patient that was not administered the recombinant or synthetic TLR5 agonist.

### Subjects

The methods provided herein can be used with a patient that is a mammal, including humans and non-human mammals. Non-human mammals treated using the present methods include domesticated animals (i.e., canine, feline, murine, rodentia, and lagomorpha) and agricultural animals (bovine, equine, ovine, porcine). In various examples, the individual to whom a compound or composition is administered is an individual who is at risk for, is suspected of having or has been diagnosed with an age-related disease or disorder.

In various embodiments of the present invention, the patient is a young human, a middle-aged human, or an elderly or geriatric human. For example, in some embodiments, the patient is between about 18 and about 35 years, or between about 18 and about 30 years, or between about 18 and about 25 years, or between about 18 and about 20 years. In some embodiments, the patient is between about 36 and about 55 years, or between about 40 and about 55 years, or between about 45 and about 55 years, or between about 36 and about 50 years, or between about 36 and about 45 years, or between about 36 and about 40 years, or between about 40 and about 50 years old, or between about 45 and about 55 years old. In some embodiments, the patient is between about 56 and about 85 years, or between about 60 and about 85 years, or about 65 and about 85 years, or between about 70 and about 85 years, or between about 75 and about 85 years, or between 80 and about 85 years, or between 56 and about 80 years, or between 56 and about 75 years, or between 56 and about 70 years, or between 56 and about 65 years, or between 56 and about 60 years, or between about 60 years and about 80 years, or about 65 years and about 75 years. In some embodiments, the patient is greater than or equal to 65 years old.

In some embodiments, the patient is about 1, or about 2, or about 3, or about 4, or about 5, or about 6, or about 7, or about 8, or about 9, or about 10, or about 11, or about 12, or about 13, or about 14, or about 15, or about 16, or about 17, or about 18, or about 19, or about 20, or about 21, or about 22, or about 23, or about 24, or about 25, or about 26, or about 27, or about 28, or about 29, or about 30, or about 31, or about 32, or about 33, or about 34, or about 35, or about 36, or about 37, or about 38, or about 39, or about 40, or about 41, or about 42, or about 43, or about 44, or about 45, or about 46, or about 47, or about 48, or about 49, or about 50, or about 51, or about 52, or about 53, or about 54, or about 55, or about 56, or about 57, or about 58, or about 59, or about 60, or about 61, or about 62, or about 63, or about 64, or or about 65, or about 66, or about 67, or or about 68, or about 69, or about 70, or about 71, or about 72, or about 73, or about 74, or about 75, or about 76, or about 77, or about 78, or about 79, or about 80, or about 81, or about 82, or about 83, or about 84, or about 85 years old. In some embodiments, the patient is at least 55 years old. In some embodiments, the patient is at least 65 years old.

A person of skill in the art will contemplate that age ranges with respect to “young,” “middle-aged,” and “elderly” or “geriatric” definitions can vary based on geographic region, among other factors. Petry, Gerontologist 2002 February; 42(1):92-9 describes age-related definitions and is hereby incorporated by reference in its entirety.

In embodiments, the biological sex of the patient is male or female. In embodiments, the biological sex of the patient is male. In embodiments, the biological sex of the patient is female.

In embodiments, the biological sex of the patient is male and the patient is middle-aged (e.g. between about 36 and about 55 years, or between about 40 and about 55 years, or between about 45 and about 55 years, or between about 36 and about 50 years, or between about 36 and about 45 years, or between about 36 and about 40 years, or between about 40 and about 50 years old, or between about 45 and about 55 years old, or between about 36 and about 64 years old, or between about 40 and about 64 years old, or between about 45 and about 64 years old). In some embodiments, the present methods, e.g. as applicable to a middle-aged male patient, prevent or reduce the severity of one or more frailties and age-related diseases or disorders.

In various embodiments of the present invention, the subject is a patient. In some embodiments, the patient is a middle-aged human. For example, in some embodiments, the patient is between about 36 and 64 years old. In further embodiments, the biological sex of the patient is male.

In various embodiments of the present invention, the subject is a patient. In some embodiments, the patient is geriatric. For example, in some embodiments, the patient is equal to or greater than about 65 years old. In further embodiments, the biological sex of the patient is male.

In some embodiments of the methods provided herein, the patient is a mammal. In some embodiments of the methods provided herein, the patient is a human. In certain embodiments of the methods provided herein, the patient is a male.

### Dosage, Administration and Pharmaceutical Formulation

In embodiments, a pharmaceutical preparation of TLR5 agonist is used in the various methods and, in some embodiments, it may be in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain other compatible therapeutic agents. Some pharmaceutical preparations can deliver the compounds of the disclosure in a sustained release formulation.

A of TLR5 agonist according to the invention, the dosage form may optionally be a liquid dosage form. Solutions can be prepared in water suitably mixed with a surfactant such as hydroxypropylcellulose or an emulsifier such as polysorbate. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms. Conventional procedures and ingredients for the selection and preparation of suitable formulations are described, for example, in Remington's Pharmaceutical Sciences (2003-20th edition) and in The United States Pharmacopeia: The National Formulary (USP 24 NF19) published in 1999. Formulations optionally contain excipients including, but not limited to, a buffering agents, an anti-oxidant, a stabilizer, a carrier, a diluent, and an agent for pH adjustment. The pharmaceutical forms suitable for injectable use include sterile aqueous solutions or dispersion and sterile powders for the extemporaneous preparation of sterile injectable solutions or dispersions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl, or benzyl alcohol; alkyl parabens such as methyl or propyl paraben; catechol; resorcinol; cyclohexanol; 3-pentanol; and m-cresol); low molecular weight (less than about 10 residues) polypeptides; proteins such as serum, albumin, gelatin, or immunoglobulins; hydrophilic polymers such as polyvinylpyrrolidone; amino acids such as glycine, glutamine, asparagine, histidine, arginine or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN, PLURONICS or polyethylene glycol (PEG).

In treatment, the dose of of TLR5 agonist optionally ranges from about 0.0001 mg/kg to about 100 mg/kg, about 0.01 mg/kg to about 5 mg/kg, about 0.15 mg/kg to about 3 mg/kg, 0.5 mg/kg to about 2 mg/kg and about 1 mg/kg to about 2 mg/kg of the subject's body weight. In other embodiments the dose ranges from about 100 mg/kg to about 5 g/kg, about 500 mg/kg to about 2 mg/kg and about 750 mg/kg to about 1.5 g/kg of the subject's body weight. For example, depending on the type and severity of the disease or disorder, about 1 .mu.g/kg to 15 mg/kg (e.g., 0.1-20 mg/kg) of agent is a candidate dosage for administration to the patient, whether, for example, by one or more separate administrations, or by continuous infusion. A typical daily dosage is in the range from about 1 mg/kg to 100 mg/kg or more, depending on the factors mentioned above. For repeated administrations over several days or longer, depending on the condition, the treatment is sustained until a desired suppression of disease or disorder symptoms occurs. However, other dosage regimens may be useful. Unit doses can be in the range, for instance of about 5 mg to 500 mg, such as 50 mg, 100 mg, 150 mg, 200 mg, 250 mg and 300 mg. The progress of therapy is monitored by conventional techniques and assays.

In some embodiments, a TLR5 agonist, e.g. flagellin or flagellin-based agent (such as entolimod) is administered to a human patient at an effective amount (or dose) of less than about 1 μg/kg, for instance, about 0.35 to about 0.75 μg/kg or about 0.40 to about 0.60 μg/kg. In some embodiments, the dose of a flagellin or flagellin-based agent (such as entolimod) is about 0.35 μg/kg, or about 0.40 μg/kg, or about 0.45 μg/kg, or about 0.50 μg/kg, or about 0.55 μg/kg, or about 0.60 μg/kg, or about 0.65 μg/kg, or about 0.70 μg/kg, or about 0.75 μg/kg, or about 0.80 μg/kg, or about 0.85 μg/kg, or about 0.90 μg/kg, or about 0.95 μg/kg or about 1 μg/kg. In various embodiments, the absolute dose of a flagellin or flagellin-based agent (such as entolimod) is about 2 μg/subject to about 45 μg/subject, or about 5 to about 40, or about 10 to about 30, or about 15 to about 25 μg/subject. In some embodiments, the absolute dose of a flagellin or flagellin-based agent (such as entolimod) is about 20 μg, or about 30 μg, or about 40 μg.

In various embodiments, the dose of TLR5 agonist, e.g. a flagellin or flagellin-based agent (such as entolimod) may be determined by the human patient's body weight. For example, an absolute dose of a flagellin or flagellin-based agent (such as entolimod) of about 2 μg for a pediatric human patient of about 0 to about 5 kg (e.g. about 0, or about 1, or about 2, or about 3, or about 4, or about 5 kg); or about 3 μg for a pediatric human patient of about 6 to about 8 kg (e.g. about 6, or about 7, or about 8 kg), or about 5 μg for a pediatric human patient of about 9 to about 13 kg (e.g. 9, or about 10, or about 11, or about 12, or about 13 kg); or about 8 μg for a pediatric human patient of about 14 to about 20 kg (e.g. about 14, or about 16, or about 18, or about 20 kg), or about 12 μg for a pediatric human patient of about 21 to about 30 kg (e.g. about 21, or about 23, or about 25, or about 27, or about 30 kg), or about 13 for a pediatric human patient of about 31 to about 33 kg (e.g. about 31, or about 32, or about 33 kg), or about 20 μg for an adult human patient of about 34 to about 50 kg (e.g. about 34, or about 36, or about 38, or about 40, or about 42, or about 44, or about 46, or about 48, or about 50 kg), or about 30 μg for an adult human patient of about 51 to about 75 kg (e.g. about 51, or about 55, or about 60, or about 65, or about 70, or about 75 kg), or about 45 μg for an adult human patient of greater than about 114 kg (e.g. about 114, or about 120, or about 130, or about 140, or about 150 kg).

In certain embodiments, a TLR5 agonist, e.g. a flagellin or flagellin-based agent (such as entolimod) in accordance with the methods provided herein is administered subcutaneously (s.c.), intravenously (i.v.), intramuscularly (i.m.), intranasally or topically. Administration of a flagellin or flagellin-based agent (such as entolimod) described herein can, independently, be one to four times daily or one to four times per month or one to six times per year or once every two, three, four or five years. Administration can be for the duration of one day or one month, two months, three months, six months, one year, two years, three years, and may even be for the life of the human patient. The dosage may be administered as a single dose or divided into multiple doses. In some embodiments, a flagellin or flagellin-based agent (such as entolimod) is administered about 1 to about 3 times (e.g. 1, or 2 or 3 times). In some embodiments, a flagellin or flagellin-based agent (such as entolimod) is administered once.

In some embodiments of the methods provided herein, TLR5 agonist, e.g. a flagellin or flagellin-based agent (such as entolimod) is administered in one or more cycles. In certain embodiments of the methods as provided herein, a TLR5 agonist, e.g. a flagellin or flagellin-based agent (such as entolimod) is administered in one or more cycles in which a cycle involves dosing a patient once per day for one day; or once a day for two days; or once a day for three days; or once a day for four days; or once a day for five days. In certain embodiments of the methods as provided herein, a TLR5 agonist, e.g. a flagellin or flagellin-based agent (such as entolimod) is administered in one or more cycles as provided herein, and wherein no more than 5 cycles are administered per year; or no more than 3 cycles are administered per year; or no more than 2 cycles are administered per year.

Various modes of administration of a TLR5 agonist, e.g. a flagellin or flagellin-based agent (such as entolimod) are contemplated herein. In one embodiment, a TLR5 agonist, e.g. a flagellin or flagellin-based agent (such as entolimod) is administered parenterally. In some embodiments, a TLR5 agonist, e.g. a flagellin or flagellin-based agent (such as entolimod) is administered by injection, e.g. intramuscular injection. In some embodiments, a TLR5 agonist, e.g. a flagellin or flagellin-based agent (such as entolimod) is by a single intramuscular injection. In some embodiments, administration is accomplished using a kit as described herein (e.g. via a unit dose form, e.g. a pre-loaded (a.k.a. pre-dosed or pre-filled) syringe or a pen needle injector (injection pen)).

### Kits

The invention provides kits that can simplify the administration of any agent described herein. An illustrative kit of the invention comprises any composition described herein in unit dosage form. In one embodiment, the unit dosage form is a container, such as a pre-filled syringe, which can be sterile, containing any agent described herein and a pharmaceutically acceptable carrier, diluent, excipient, or vehicle. The kit can further comprise a label or printed instructions instructing the use of any agent described herein. The kit may also include a lid speculum, topical anesthetic, and a cleaning agent for the administration location. The kit can also further comprise one or more additional agent described herein. In one embodiment, the kit comprises a container containing an effective amount of a composition of the invention and an effective amount of another composition, such those described herein.

### Definitions

With respect to the agents described herein, the terms “modulate” and “modulation” refers to the upregulation (i.e., activation or stimulation) or downregulation (i.e., inhibition or suppression) of a response. A “modulator” is an agent, compound, or molecule that modulates, and may be, for example, an agonist, antagonist, activator, stimulator, suppressor, or inhibitor. The terms “inhibit”, “reduce”, remove as used herein refer to any inhibition, reduction, decrease, suppression, downregulation, or prevention in expression, activity or symptom and include partial or complete inhibition of activity or symptom. Partial inhibition can imply a level of expression, activity or symptom that is, for example, less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, or less than 5% of the uninhibited expression, activity or symptom. The terms “eliminate” or “eradicate” indicate a complete reduction of activity or symptom.

As used herein, the term “a disorder” or “a disease” refers to any derangement or abnormality of function; a morbid physical or mental state. See Dorland's Illustrated Medical Dictionary, (W.B. Saunders Co. 27th ed. 1988).

As used herein, the term “treating” or “treatment” of any disease or disorder refers in one embodiment, to ameliorating the disease or disorder (i.e., slowing or arresting or reducing the development of the disease or at least one of the clinical symptoms thereof). In another embodiment “treating” or “treatment” refers to alleviating or ameliorating at least one physical parameter including those which may not be discernible by the patient. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to preventing or delaying the onset or development or progression of the disease or disorder.

“Therapeutically effective amount” as used herein means the amount of a compound or composition (such as described herein) that causes at least one desirable change in a cell, population of cells, tissue, individual, patient or the like. In some embodiments a therapeutically effective amount as used herein means the amount of a compound or composition (such as described herein) that prevents or provides a clinically significant change in a disease or disorder or condition (e.g., reduce by at least about 30 percent, at least about 50 percent, or at least about 90 percent) or in one or more features of a disease or disorder or condition described herein.

### EXAMPLES

The present disclosure will be further described in the following examples, which do not limit the scope of any invention or inventions described in the claims.

### Example 1: Co-Administration with Entolimod Improves Vaccination Efficacy

This example describes the use of a pharmacological flagellin-based agent for improving or increasing immune response in vaccinated mice, as compared to vaccinated mice that did not receive the flagellin-based agent.

Specifically, the impact of entolimod on vaccination efficacy in male mice at different ages was evaluated. As shown in Table 1, groups of young (30 weeks), middle-aged (73 weeks), and old (113 weeks) male NIH Swiss mice were vaccinated with Prevnar13 pneumococcus vaccine (0.125 μg), alone or in combination with a dose of entolimod at 0.1 μg/mouse or 1.0 μg/mouse administered via subcutaneous administration. A control dose of PBS was administered instead of entolimod in control groups.

TABLE 1
Study Design of Vaccine Efficacy Experiment with Entolimod

| | | | Immunization on Days 1 | | |
|---|---|---|---|---|---|
| | | | and 15 | | |
| | No. of mice | Age at start of | Prevnar13 | Entolimod | |
| | (male NIH | experiment | dose | dose | |
| Group No. | Swiss) | (weeks) | (μg/mouse) | (μg/mouse) | Evaluation |
| 1 | 5 | 30 | 0.125 | N/A* | Serum collection |
| 2 | 5 | 30 | 0.125 | 0.1 | on Days 0, 12, |
| 3 | 5 | 30 | 0.125 | 1.0 | and 27. Sacrifice |
| 4 | 5 | 73 | 0.125 | N/A* | and spleen |
| 5 | 5 | 73 | 0.125 | 0.1 | collection on Day |
| 6 | 5 | 73 | 0.125 | 1.0 | 27. |
| 7 | 5 | 113 | 0.125 | N/A* | |
| 8 | 6 | 113 | 0.125 | 0.1 | |
| 9 | 6 | 113 | 0.125 | 1.0 | |

*PBS administered instead of entolimod

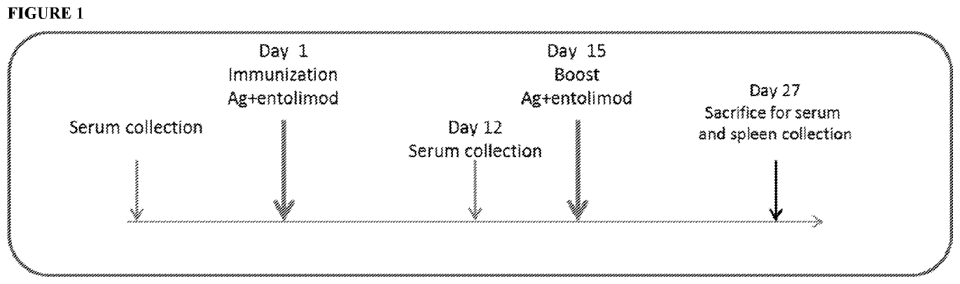

The mouse dose of vaccine was previously determined using the Food and Drug Administration (FDA) human dose equivalent surface area-based formula (Haas, J Infect Dis. 2014 Jan. 1; 209(1): 87-97, the content of which is hereby incorporated by reference in its entirety). Mice in all groups were vaccinated on Day 1 of the experiment and given a booster injection on Day 15. Serum samples were collected prior to the first immunization (“Day 0”, baseline) and on Days 12 and 27. The experiment was terminated on Day 27, at which point mice were euthanized and spleens were collected for analysis for T-cell populations. A schematic presentation of the timeline of the experiment is displayed in FIG. 1.

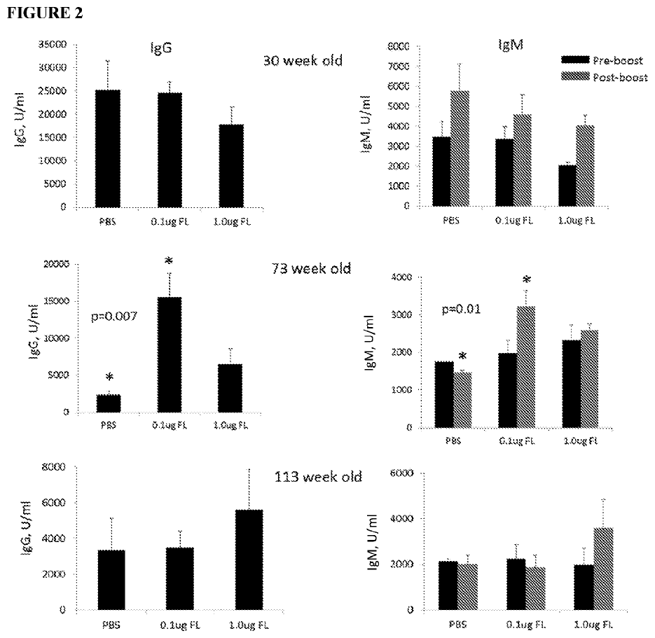

To assess the efficacy of vaccination in each group of mice, serum samples were tested for levels of pneumococcus/Prevnar13-specific IgG and IgM antibodies using commercially available ELISA kits, and the results are depicted in FIG. 2. Levels of pneumococcus/Prevnar13-specific IgG (left panels) and IgM (right panels) were measured by ELISA in serum samples collected on Day 12 or Day 27 after immunization on Day 1 (with booster immunization given on Day 15). Mean antibody titers are shown, with * indicating statistically significant differences as determined by Student's t-test. All other comparison between matched PBS-treated and entolimod-treated groups were not statistically significant (P>0.3). In the figures, FL is synonymous with entolimod for this experiment.

Administration of entolimod along with Prevnar13 vaccine did not show a boost in antibody production in old mice (113 weeks) at either tested concentration. Young (30 week old) mice were much more responsive to vaccination, producing very high titers specific IgG and IgM (10-fold higher than the levels observed in old animals), but co-administration of entolimod did not cause any further increase. Increased immune response was detected, however, in the middle-aged (73 weeks) mouse cohort. All of the middle-aged mice responded to immunization with the Prevnar13 vaccine, but the titers of induced antibodies were very low, similar to those produced by old mice. These antibody levels were significantly boosted, however, by co-administration of entolimod with the vaccine (˜7-fold for IgG and ˜2-fold for IgM for groups treated with 0.1 μg/mouse entolimod). Interestingly, the lower concentration of entolimod tested in this experiment (0.1 μg/mouse) was more effective than the higher concentration (1.0 μg/mouse).

### Example 2: Co-Administration with Entolimod Stimulates Effectors of Immune Response

This experiment evaluated the effect of entolimod administration on the number and phenotype of T cells activated in response to vaccination.

Spleens of mice treated on Day 27 after initial immunization were collected and a single-cell suspension of total spleen cells was prepared and stained with antibodies against the lymphocyte and T cell markers. Absolute numbers and percentages of different cell populations were determined by flow cytometry. Specifically, 100 μL (1×10⁶ cells) of the cell suspension was aliquoted into 5 mL polystyrene round bottom tubes. Cells were then stained for CD3e-BV421 (clone 145-2C11, BD Biosciences, 562600), CD4-APC (clone RM4.5, BD Biosciences, 553051), CD8-BB700 (clone 53-6.7, BD Biosciences, 566409) and CD44-BB515 (clone IM7, BD Biosciences, 564587), alongside a viability dye (LiveDead Fixable Aqua, ThermoFisher, L34957). Cells were mixed by vortexing briefly and incubated for 20 minutes at room temperature while also being protected from light. Following staining, red blood cells were lysed with 3 mL of ACK lysing buffer (ThermoFisher, A1049201) and incubated for 10 minutes at room temperature. Cells were pelleted by centrifugation, supernatants discarded, and cells were washed with 3 mL of wash buffer (1×DPBS+0.5% BSA). Washed cells were pelleted by centrifugation and supernatants discarded. Cell pellets were resuspended by vortexing briefly and fixed with 100 μL of 2% formaldehyde in PBS and stored in the dark at 4° C. until acquisition.

Data were acquired on an LSRFortessa instrument (Becton Dickinson, San Jose, Calif., USA). One hundred thousand events were collected and compensation was performed after acquisition. FCS files were analyzed using WinList version 8.0 software (Verity Software House). Cells were hierarchically gated based on single events (forward scatter area versus height) and lymphocytes (forward scatter area versus side scatter area). CD3+ events were selected and from this CD4+ and CD8+ T cells were selected. The percentage positive as well as absolute numbers in these populations were recorded, and lastly, CD44 expression and absolute numbers were recorded for each T cell subset.

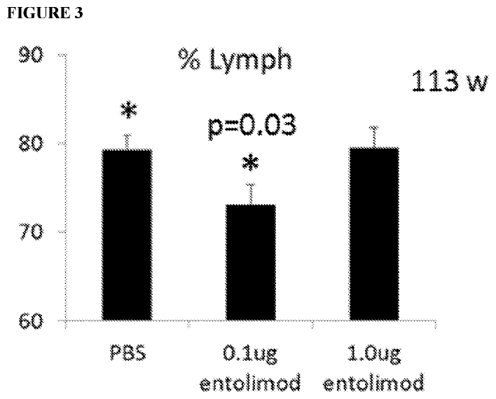

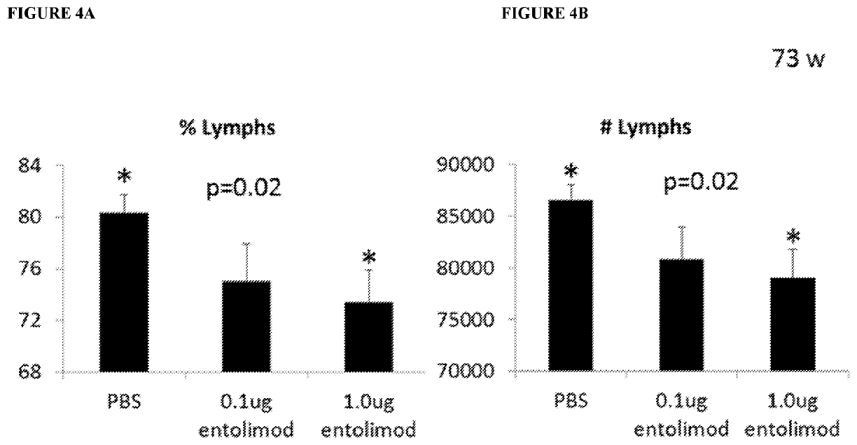

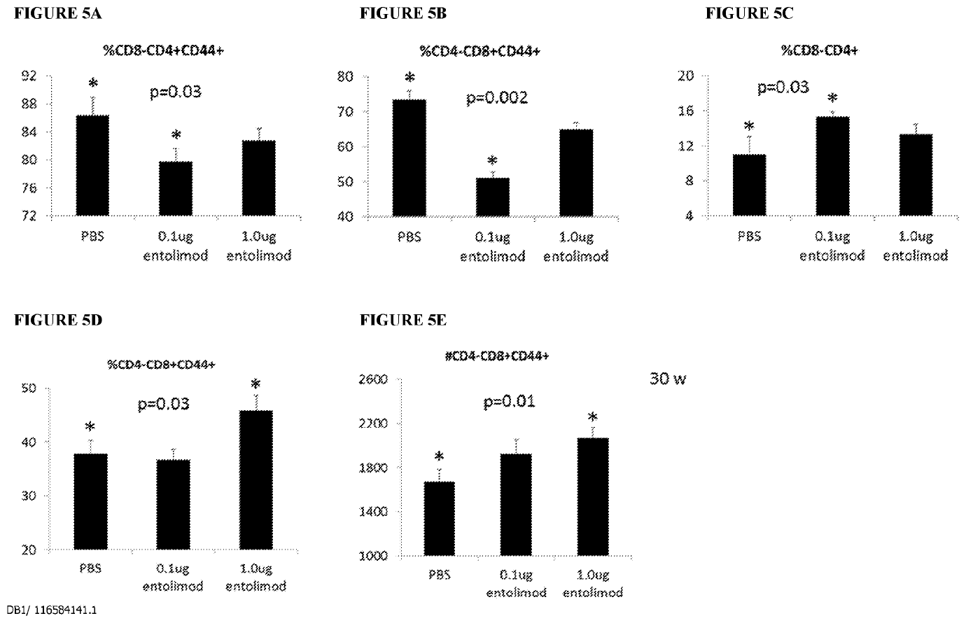

FIG. 3 depicts the percentage of lymph cells in a single-cell suspension of total spleen cells from mice that were 113 weeks old at the time of co-administration of Prevnar13 and entolimod (at 0.1 μg/mouse and 1.0 μg/mouse) or PBS. FIG. 4a-b depicts both the (a) percentage and (b) total number of lymph cells in a single-cell suspension of spleen cells from mice that were 73 weeks old at the time of co-administration of Prevnar13 and entolimod (at 0.1 μg/mouse and 1.0 μg/mouse) or PBS. FIG. 5a-e depicts percentages and number of various T cell markers (e.g., CD8−, CD4+, and CD44+) in a single-cell suspension of spleen cells from mice that were 30 weeks old at the time of co-administration of Prevnar13 and entolimod (at 0.1 μg/mouse and 1.0 μg/mouse) or PBS.

### Example 3: Co-Administration of Entolimod with Vaccine Improved Vaccination Efficacy Despite Varying Routes of Administration and Formulations

The purpose of this study was to evaluate whether entolimod's effect as an immunostimulator is dependent upon the route of administration and/or formulation used.

A comparison was drawn between the efficacy of immunization of middle-aged male (aged 73 weeks) NIH Swiss mice with Tdap vaccine when the vaccine was given alone or in combination with entolimod delivered by different routes of administration and in different formulations: through intramuscular injection mixed with vaccine; through subcutaneous injection; or through subcutaneous injection mixed with an adjuvant (Imject Alum, Thermo Scientific).

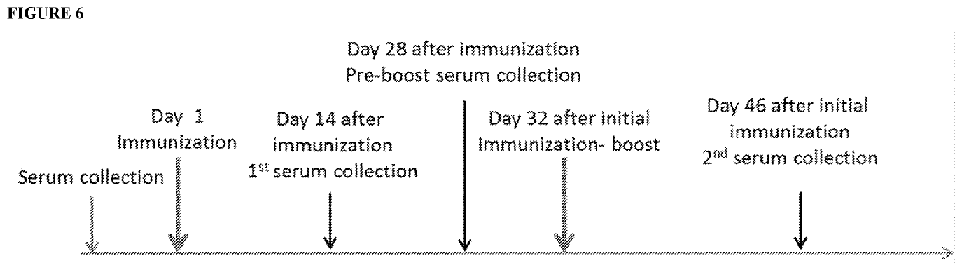

Boostrix is the Tdap vaccine that was used. Boostrix is an FDA-approved vaccine used in humans to protect against Tetanus, Diphtheria and Acellular Pertussis. The Tdap vaccine was delivered to mice in this experiment at one fourth of the approved human dose. FIG. 6 shows a schematic illustration of the immunization schedule. Mice were immunized on Day 1 of the experiment and received a booster immunization on Day 32. Serum samples were collected on Day 0 (baseline), Day 14, Day 28, and Day 46.

Titers of tetanus-specific IgG antibodies in serum samples collected during the course of the experiment were measured by ELISA. The data presented in FIG. 7 show that Tdap vaccination of middle-aged mice was effective and that administration of entolimod along with the vaccine significantly increased the efficacy of vaccination about 2-fold (P=0.015 by one-way ANOVA test) as compared to administration of the vaccine alone. It was also found that the beneficial effect of entolimod did not depend on either the route of entolimod administration or the presence of alum as an adjuvant.

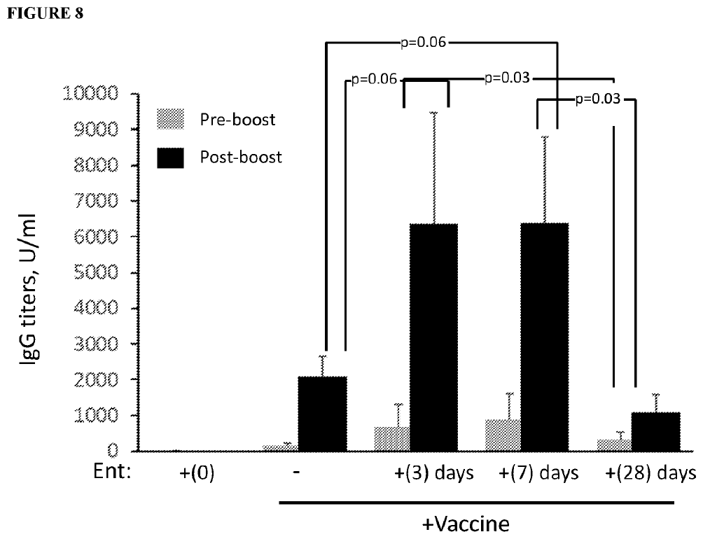

A comparison was drawn between the efficacy of immunization of geriatric male (aged 83 weeks) mice with Prevnar13 vaccine when the vaccine was given 3 days after, 7 days after, or 28 days after subcutaneous entolimod administration. Specifically, 83-weeks-old mice received 0.3 μg of Entolimod via subcutaneous administration and were vaccinated with Prevnar13 vaccine 3 days, 7 days, or 28 days later (labeled as “+(3)”, “+(7)” and “+(28)”). Control groups received vaccine only (labeled as “(−)”) or Entolimod only (labeled as “+(0)”). All groups received boost (injection of vaccine alone) 14 days after initial vaccination. Serum was collected 14 days after initial vaccination (pre-boost) and 14 days after boost (post-boost). IgG titers were measured using commercial IgG ELISA kit. FIG. 8 depicts increased IgG titer measurements post-boost in the +3 days and +7 days vaccine administration groups, as compared to control groups.

### Example 4: Immunogenicity, Pharmacodynamics, and Safety of Co-Administration of Entolimod with Influenza Vaccine in Geriatric Patients

A randomized, double-blind, placebo-controlled, single-administration, sequential-group, dose-ranging study evaluating the immunogenicity, pharmacodynamics, and safety of entolimod within the geriatric population (≥65 years old) vaccinated against influenza is performed.

The primary objectives of the study are threefold: (1) to evaluate the effect of increasing dose of entolimod's on enhancement of the influenza vaccine immunogenicity in the geriatric population (≥65 years old); (2) to characterize the safety profile of entolimod within the geriatric population (≥65 years old) vaccinated against influenza; and (3) to evaluate the pharmacodynamic effect of entolimod on immunological status, frailty index (FI) and quality of life (QoL).

### Overall Study Setup

A total of 100 individuals are randomized into one of four treatment groups (placebo and three progressive dosage entolimod groups).

After providing a written informed consent, subjects undergo screening medical history, physical examination, vital signs, laboratory, and electrocardiogram (ECG) assessments within 7 days prior to study drug administration and influenza vaccination.

Eligible subjects receive the influenza vaccination (Fluzone, high-dose split virion influenza virus vaccine, Sanofi Pasteur) and a single intramuscular (IM) injection of the study drug (entolimod or placebo). For safety assessment, three sequential cohorts of subjects are enrolled at progressively higher entolimod dose levels of 1 μg, 3 μg, and 10 μg. Initial safety assessment is done in the first 4 subjects (3 receive entolimod and 1 receives placebo) within each dose group. If ≤1 subject experiences dose-limiting toxicities (DLTs) over a period of 7 days post vaccination, dose expansion proceeds for that group by enrolling a total of 25 subjects (20 to receive entolimod and 5 to receive placebo). Dose escalation proceeds step-wise to the next planned dose following enrollment completion of the previous group. If 2 DLTs are observed within a dose group, the study information from those individuals will be unblinded. If it is found that the subjects exhibiting DLTs were treated with the study drug entolimod, the clinical trial ethics committee will be engaged for study evaluation, and a determination made about the continuing dosing of subjects.

Reference is made to the Guidance for Industry Toxicity Grading Scale for Healthy Adult and Adolescent Volunteers Enrolled in Preventive Vaccine Clinical Trials for grading the severity of adverse events (AEs) and laboratory abnormalities where applicable. Based on the known safety profile of entolimod, flu-like symptoms are expected. Decreases in blood pressure, increases in heart rate, hepatic transaminase elevations, hyperglycemia, and hypophosphatemia are expected. A dose-limiting toxicity (DLT) is defined as any of the treatment emergent adverse events (TEAE5) or treatment-emergent laboratory abnormalities based on the sound clinical judgment of the investigator and an independent safety monitor.

Subjects are evaluated at the study center on the day of study drug administration (Day 1), for ≥6 hours (2 hrs, 4 hrs and 6 hrs), and thereafter on weeks 1 and 4, and then on months 1, 2, 6 and 12. Between months 2 and 6 and 6 and 12, adverse events are reported and occurrence of respiratory infections is assessed via phone interview. Assessments of adverse events, vital signs/oxygen saturation, clinical chemistry and hematology parameters, ECGs, plasma cytokines, leukocytes, anti-A/H1N1, anti-A/H3N2, and anti-B influenza serum circulating antibodies including cellular immune response outcomes are performed to describe drug safety, pharmacodynamics, and immunogenicity.

### Study Drug Administration

### Study Drug Dosage Forms, Strength, and Stability

Study drugs (entolimod and placebo) are manufactured under current Good Manufacturing Practices (cGMP).

Entolimod is provided as a liquid for intramuscular (IM) injection in 2 mL prefilled, single-use vials containing 50 μg of entolimod in 0.5 mL (concentration of 100 μg/mL) of formulation. The formulation comprises phosphate-buffered saline (PBS) containing 0.1% polysorbate 80 (Tween 80).

A matching placebo is also provided. The placebo has the same excipient composition as the active drug formulation (ie, PBS containing 0.1% Tween 80) and fill-finished in the same type of 2 mL single use vials (0.5 mL per vial).

Study drug and placebo are stored at −70±10° C. Once thawed, the drug is kept refrigerated and administered within 8 hours.

### Study Drug Injection

A single IM injection of study drug (entolimod or placebo) is administered to each subject on the morning of vaccination, Day 1 (to be administered within 1 minute of flu vaccine, in the same location). The appropriate amount of study drug is aseptically withdrawn from the required number of study drug vials into a 1-mL tuberculin syringe calibrated in 10-μL units. The drug is administered from the syringe through a 1.5-inch, 25-gauge needle at a 90°-angle to the skin surface into the deltoid muscle between the acromion process and the midaxillary line.

### Dose Randomization and Allocation

Study subjects are randomized within dosing cohort to entolimod or placebo; the intent being to provide a contemporaneously accrued group of control subjects to provide context for the results observed in subjects who receive entolimod. The study biostatistician provides a coded randomization list to the study center pharmacist indicating dosing assignments for subjects within each dosing cohort. Subjects and study center personnel involved in the care of subjects are blinded to study drug assignment (entolimod vs. placebo) but not to dose or injection volume. Given the exploratory nature of the study, the study sponsor is unblinded as to study drug assignment but does not convey this information to study center personnel unless or until necessary for subject safety or dose-escalation decisions.

Cohorts of subjects are sequentially enrolled at progressively higher starting dose levels. The following dose levels are planned: (1) dose Level 1: 1 μg; (2) dose Level 2: 3 μg; and (3) dose Level 3: 10 μg. These doses are within the dose range of 1 ug to 50 μg and equal or greater than five times less than that which has been previously evaluated in healthy subjects and patients with advanced solid tumors.

### Primary Endpoints

Immunogenicity is measured as a primary endpoint as a proportion of patients who achieved seroconversion. Seroconversion to the influenza virus vaccine is defined by either a four-fold increase in the antibody titers between the pre-vaccination and at Day 30, or an increase of antibody titers from <1:10 to ≥1:40 for pre-vaccination and the Day 30 serum samples.

Immunogenicity is also measured as a primary endpoint by changes of the anti-A/H1N1, anti-A/H3N2, and anti-B influenza virus strains serum circulating antibodies (as assessed using enzyme-linked immunosorbent assay (ELISA) or hemagglutination inhibition (HAI) assay) levels.

Safety is measured as a primary endpoint as treatment-emergent adverse events (TEAE5); laboratory abnormalities; oxygen saturation and vital sign changes, adverse electrocardiogram (ECG) findings.

### Secondary Endpoints

Pharmacodynamics and cellular immune responses to influenza vaccination are assessed as secondary endpoints. For example, these secondary endpoints are measured by the following: (1) time to onset and the number of upper-respiratory infections, including (but not limited to) influenza viral infections (as indicated by subject self-reporting); (2) changes in the concentration of circulating plasma cytokines including (but not limited to): IL-6 and G-CSF as pharmacodynamic indicators of entolimod's activity (measured using ELISA); (3) changes to 10 cytokine/chemokine mediators of adaptive immune function (IFN-γ, IL-1β, IL-2, IL-4, IL-6, IL-8, IL-10, IL-12p70, IL-13 and TNF-α) in PBMC culture supernatants at 0 and 24 hours after influenza (A/H1N1, A/H3N2 and B) viral stimulation as detected by Meso Scale Discovery, V-PLEX Proinflammatory panel human immunoassay kits; (4) changes in the quantification of IFNγ-positive cells as a marker of cell-mediated immunity (CMI) after vaccination using influenza virus-specific IFN-γ ELISPOT assay kits from R&D Systems; and (5) comparison between the frailty index (FI) and quality of life (QoL) of individuals receiving entolimod versus placebo (as measures from patient self-reporting questionnaires).

### Statistical Methods

Appropriate data analysis sets are defined. The full-analysis set includes data from all randomized subjects. The safety analysis set includes data from all randomized subjects who receive study drug (entolimod or placebo). Evaluable analysis sets are defined and include data from subjects who have the necessary baseline and on-study measurements to provide interpretable results for specific parameters of interest.

Data is described and summarized by study drug assignment (entolimod or placebo), dose level, and time point. As appropriate, changes from baseline to each subsequent time point is described and summarized. Similarly, as appropriate, the most extreme change from baseline during the study is described and summarized. Shift tables or graphical techniques (eg, bar charts, line graphs) are used when such methods are appropriate and informative. Descriptive summaries include sample size; mean; standard deviation; 95% confidence intervals (CIs) on the mean, median, minimum, and maximum for continuous variables and counts; percentages; and 95% CIs on the percentage for categorical variables.

Analyses are based upon the observed data unless methods for handling missing data are specified. If there is a significant degree of non-normality, analyses may be performed on log-transformed data or nonparametric tests may be applied, as appropriate.

Statistical testing is 2-sided at a nominal 0.05 level of significance. Given the exploratory nature of this study, adjustments for multiple comparisons are not applied.

Based on the full-analysis set, information regarding subject demographics, comorbidities, treatment history, and other baseline characteristics are described.

Study drug administration, concomitant medication use, supportive care use, AEs, laboratory abnormalities, vital signs/oxygen saturation, body weight, virology data, quality of life and frailty index status are described and summarized. For safety analyses, AEs are classified using the Medical Dictionary for Regulatory Activities (MedDRA). The severity of AEs are graded by the investigator according to the Guidance for Industry Toxicity Grading Scale for Healthy Adult and Adolescent Volunteers Enrolled in Preventive Vaccine Clinical Trials. Concomitant medication use is coded using the World Health Organization Drug Dictionary (WHODRUG) into Anatomical Therapeutic-Chemical classification (ATC) codes; these data descriptions particularly focus on supportive medications and care provided in response to any study-study-induced adverse effects and to therapies for GVHD. For ECG assessment of QT intervals, correction by both the Bazett and Fridericia methods is applied, and the data is summarized by CTCAE grading categories in terms of absolute QTc and maximal QTc change from baseline.

Pharmacodynamic measures are listed and summarized using appropriate graphical and tabular methods. Statistical analysis evaluating pharmacodynamic parameters is performed using contrasts in the context of analysis of covariance (ANCOVA).

The number of upper-respiratory infection events, time to onset, and the length of time for the active viral infection are noted using the appropriate tabular methods and analyzed based on statistical ANCOVA among the study drug and placebo groups.

Using appropriate regression or stratification techniques, associations between subject characteristics (eg, sex, race, age, weight, dose) and outcome measures (eg, pharmacodynamic parameters) are explored. Similarly, associations between outcome measures (eg, relationships between pharmacodynamic parameters) are evaluated.

### EQUIVALENTS

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections.

As used herein, all headings are simply for organization and are not intended to limit the disclosure in any manner. The content of any individual section may be equally applicable to all sections.

What is claimed is:

1. A method of improving vaccine efficacy in a patient, said method comprising administering to the patient in need thereof a recombinant or synthetic TLR5 agonist and an antigen which stimulates an immune response against a disorder,
wherein the immune response is enhanced or promoted in the patient relative to the immune response of a patient that was not administered the recombinant or synthetic TLR5 agonist.

2. A method of improving vaccine efficacy in a patient, said method comprising administering to the patient in need thereof an antigen which stimulates an immune response against a disorder, wherein said patient has received or is receiving a recombinant or synthetic TLR5 agonist,
wherein the immune response is enhanced or promoted in the patient relative to the immune response of a patient that was not administered the recombinant or synthetic TLR5 agonist.

3. A method of improving vaccine efficacy in a patient, said method comprising administering to the patient in need thereof a recombinant or synthetic TLR5 agonist, wherein said patient has received or is receiving an antigen which stimulates an immune response against a disorder,
wherein the immune response is enhanced or promoted in the patient relative to the immune response of a patient that was not administered the recombinant or synthetic TLR5 agonist.

4. The method of any one of the above claims, wherein the immune response is enhanced or promoted before the patient is administered the antigen.

5. The method of any one of claims 1-3, wherein the immune response is enhanced or promoted following administration of the antigen to the patient.

6. The method of any one of the above claims, wherein the patient is immunosenescent.

7. The method of any one of the above claims, wherein the patient has an impaired immune system.

8. The method of any one of the above claims, wherein the patient is immunocompromised.

9. The method of claim 1, wherein the patient is middle-aged.

10. The method of claim 9, wherein the patient is between about 36 and about 64 years old.

11. The method of claim 1, wherein the patient is geriatric.

12. The method of claim 11, wherein the patient is equal to or older than about 65 years old.

13. The method of claim 12, wherein the patient has an age-related immune system impairment.

14. The method of any one of the above claims, wherein the biological sex of the patient is male.

15. The method of any one of claims 1-13, wherein the biological sex of the patient is female.

16. The method of any one of the above claims, wherein the TLR5 agonist is entolimod or a derivative thereof.

17. The method of claim 16, wherein the TLR5 agonist comprises a polypeptide having an amino acid sequence having at least 95% sequence identity to SEQ ID NO: 1.

18. The method of claim 17, wherein the TLR5 agonist comprises a polypeptide having the amino acid sequence that is SEQ ID NO: 1.

19. The method of claim 16, wherein the TLR5 agonist comprises a polypeptide having an amino acid sequence having at least 95% sequence identity to SEQ ID NO: 4.

20. The method of claim 19, wherein the TLR5 agonist comprises a polypeptide having the amino acid sequence of SEQ ID NO: 4.

21. The method of claim 16, wherein the TLR5 agonist comprises a polypeptide having an amino acid sequence having at least 95% sequence identity to one of SEQ ID NOs: 2-3, 5-26, and 28.

22. The method of claim 21, wherein the TLR5 agonist comprises a polypeptide having the amino acid sequence of one of SEQ ID NOs: 2-3, 5-26, and 28.

23. The method of any one of the above claims, wherein titer levels of antigen-specific antibodies are higher as compared to titer levels of antigen-specific antibodies in patients that were not administered the TLR5 agonist.

24. The method of claim 23, wherein the titer levels of antigen-specific antibodies are at least about 2-fold higher as compared to titer levels of antigen-specific antibodies in patients that were not administered the TLR5 agonist.

25. The method of any one of the above claims, wherein the patient's innate immune response is increased as compared to the innate immune response of a patient that was not administered the TLR5 agonist.

26. The method of any one of the above claims, wherein the patient's adaptive immune response is increased as compared to the adaptive immune response of a patient that was not administered the TLR5 agonist.

27. The method of any one of the above claims, wherein the patient's innate immune response and adaptive immune response are increased as compared to the innate and adaptive immune responses of a patient that was not administered the TLR5 agonist.

28. The method of any one of the above claims, wherein the patient's T cell population(s) are increased and/or restored as compared to the T cell populations of a patient that was not administered the TLR5 agonist.

29. The method of any one of the above claims, wherein the patient's T cells, including T cells selected from one or more of CD4+ effector T cells, CD8+ effector T cells, CD4+ memory T cells, CD8+ memory T cells, CD4+ central memory T cells, CD8+ central memory T cells, natural killer T cells, CD4+ helper cells, and CD8+ cytotoxic cells, are increased and/or restored as compared to the T cell populations of a patient that was not administered the TLR5 agonist.

30. The method of any one of the above claims, wherein the method results in a reduction of vaccine dosage, relative to the vaccine dosage of a patient that was not administered the recombinant or synthetic TLR5 agonist.

31. The method of any one of the above claims, wherein the method results in a reduction of the frequency of vaccine dosing, relative to the frequency of vaccine dosing of a patient that was not administered the recombinant or synthetic TLR5 agonist.

32. The method of any one of the above claims, wherein the antigen is a constituent of an infectious agent selected from a live and attenuated, killed, inactivated, and toxoid infectious agent.

33. The method of any one of the above claims, wherein the antigen is associated with a disease and/or disorder selected from tetanus, diphtheria, acelluar pertussis, and invasive infection caused by pneumococcal bacterial species.

34. The method of claim 33, wherein the invasive infection caused by pneumococcal bacterial species is pneumonia or meningitis.

35. The method of claim 33, wherein the disease and/or disorder is tetanus, diphtheria and/or pertussis.

36. The method of any one of claims 1-32, wherein the antigen is associated with a viral infection.

37. The method of claim 36, wherein the viral infection is influenza.

38. The method of any one of claims 1-31, wherein the antigen is associated with one or more of a tumor cell, a cell with damaged DNA, and a senescent cell.

39. The method of any one of the above claims, wherein the recombinant TLR5 agonist is administered at a dose of between about 0.5 μg to about 15 μg.

40. The method of any one of the above claims, wherein the recombinant TLR5 agonist is not administered with an adjuvant.

41. The method of the previous claim, wherein the adjuvant is selected from one or more of alum, AS04, AS03, aluminum hydroxide, aluminum phosphate, and potassium aluminum sulfate.

42. A TLR5 agonist for use in improving vaccine efficacy in a patient, comprising a recombinant TLR5 agonist and an antigen which stimulates an immune response against a disorder,
wherein the immune response is enhanced or promoted in the patient.

43. A method of improving vaccine efficacy in a geriatric and immunosenescent patient, said method comprising administering to the geriatric and immunosenescent patient in need thereof a recombinant TLR5 agonist and an antigen which stimulates an immune response against a disorder,
wherein the immune response is enhanced or promoted in the patient relative to the immune response of a patient that was not administered the recombinant TLR5 agonist,
wherein vaccine efficacy is improved relative to an expected age-related response in a geriatric patient that was not administered the recombinant TLR5 agonist.

44. The method of claim 43, wherein the patient is equal to or older than about 65 years old.

45. The method of claim 43, wherein the patient has an age-related immune system impairment.

46. The method of claim 43, wherein the biological sex of the patient is male or female.

47. The method of claim 43, wherein the TLR5 agonist is entolimod or a derivative thereof,
wherein the TLR5 agonist comprises a polypeptide having an amino acid sequence having at least 95% sequence identity to or an amino acid sequence that is SEQ ID NO: 1.

48. The method of claim 43, wherein the TLR5 agonist comprises a polypeptide having an amino acid sequence having at least 95% sequence identity to or the amino acid sequence of SEQ ID NO: 4.

49. The method of claim 43, wherein the TLR5 agonist comprises a polypeptide having an amino acid sequence having at least 95% sequence identity to or the amino acid sequence of one of SEQ ID NOs: 2-3, 5-26, and 28.
